SEQUENCE LISTING (9 sequences)

SEQ ID NO: 1 - DNA, 30 bp, synthetic construct
  misc_feature 1..30: note = Description of sequence: oligonucleotide, name: DBCO-Apt aptamer sequence; S2.2 sequence
  source 1..30: mol_type = other DNA
  modified_base 1: mod_base = OTHER; note = thymine-dibenzocyclooctyne
tttttgcagttgatcctttggataccctgg

SEQ ID NO: 2 - DNA, 30 bp, synthetic construct; identical to SEQ ID NO: 1
  misc_feature 1..30: note = Description of sequence: oligonucleotide, name: FAM-Apt-DBCO aptamer sequence
  source 1..30: mol_type = other DNA
  modified_base 1: mod_base = OTHER; note = thymine-dibenzocyclooctyne
  modified_base 30: mod_base = OTHER; note = guanine-fluorescein

SEQ ID NO: 3 - DNA, 30 bp, synthetic construct; identical to SEQ ID NO: 1
  misc_feature 1..30: note = Description of sequence: oligonucleotide, name: NH2-Apt aptamer sequence
  source 1..30: mol_type = other DNA
  modified_base 1: mod_base = OTHER; note = thymine-NH2

SEQ ID NO: 4 - DNA, 30 bp, synthetic construct; identical to SEQ ID NO: 1
  misc_feature 1..30: note = Description of sequence: oligonucleotide, name: FAM-Apt-NH2 aptamer sequence
  source 1..30: mol_type = other DNA
  modified_base 1: mod_base = OTHER; note = thymine-NH2
  modified_base 30: mod_base = OTHER; note = guanine-fluorescein

SEQ ID NO: 5 - DNA, 56 bp, synthetic construct
  misc_feature 1..56: note = Description of sequence: oligonucleotide, name: 6H-Tag1 oligonucleotide sequence
  source 1..56: mol_type = other DNA
gtacctttctattctcactctcatcatcatcatcatcattcctccaaactgcagtc

SEQ ID NO: 6 - DNA, 56 bp, synthetic construct
  misc_feature 1..56: note = Description of sequence: oligonucleotide, name: 6H-Tag2 oligonucleotide sequence
  source 1..56: mol_type = other DNA
ggccgactgcagtttggaggaatgatgatgatgatgatgagagtgagaatagaaag

SEQ ID NO: 7 - DNA, 102 bp, synthetic construct
  misc_feature 1..102: note = Description of sequence: oligonucleotide, name: Y1 oligonucleotide sequence for Y-shaped DNA scaffold
  source 1..102: mol_type = other DNA
cacgcatagcctttgctcctcgtctggaacgtcgcagctttagttctgggcctatgcgtgttttttgtagtcggtacctaagacttctgagcatgcactgac

SEQ ID NO: 8 - DNA, 86 bp, synthetic construct
  misc_feature 1..86: note = Description of sequence: oligonucleotide, name: Y2 oligonucleotide sequence for Y-shaped DNA scaffold
  source 1..86: mol_type = other DNA
cactacagaggttgcgtctgtcccacgttgtcatggggggttggcctgttttttgtcagtgcatgctcagaagaactcacgtgacg

SEQ ID NO: 9 - DNA, 45 bp, synthetic construct
  misc_feature 1..45: note = Description of sequence: oligonucleotide, name: Y3 oligonucleotide sequence for Y-shaped DNA scaffold
  source 1..45: mol_type = other DNA
  modified_base 1: mod_base = OTHER; note = adenine-dibenzocyclooctyne
attgcgtatgtcacgtcacgtgagttcgtcttaggtaccgactac

(19) United States
(12) Patent Application Publication
(10) Pub. No.: US 2026/0056202 A1
(43) Pub. Date: Feb. 26, 2026

(54) USE OF MODIFIED PHAGE FOR CAPTURING AND SUBTYPING OF CANCER CELLS

(71) Applicant: The Chinese University of Hong Kong, Sha Tin (CN)

(72) Inventor: Chuanbin MAO, Sha Tin (CN)

(21) Appl. No.: 19/261,867

(22) Filed: Jul. 7, 2025

Related U.S. Application Data
(60) Provisional application No. 63/668,574, filed on Jul. 8, 2024.

(51) Int. Cl. G01N 33/574; C12N 7/00; C12N 15/115; G01N 33/543

(52) U.S. Cl. CPC G01N 33/57492; C12N 7/00; C12N 15/115; G01N 33/54326; C12N 2795/14121; C12N 2795/14122

(57) ABSTRACT

The present disclosure relates to genetically engineered phage that bind to cancer cells. The genetically engineered phage can be used to isolate cancer cells, for example, circulating cancer cells, by binding to antigens specifically expressed on the surface of cancer cells. Thus, the present disclosure provides compositions, methods, and kits for improved cancer diagnosis that are useful for improved cancer treatment.

### CROSS-REFERENCE TO RELATED APPLICATIONS

This application claims the benefit of priority to U.S. Provisional Application No. 63/668,574, filed Jul. 8, 2024, the contents of which are incorporated herein by reference in its entirety.

### REFERENCE TO A SEQUENCE LISTING

The instant application contains a sequence listing which has been submitted electronically in XML file format and is hereby incorporated by reference in its entirety. The XML copy of the sequence listing “080015-1509212-041310US_ST26.xml,” was created on Nov. 11, 2025, is 16,867 bytes in size.

### BACKGROUND

Circulating tumor cells (CTCs) are cells in the bloodstream that originate from solid tumors. CTCs hold significant promise as targets for liquid biopsy in diagnosing tumor metastases and assessing prognosis (Fidler, I. J. Nat Rev Cancer. 3, 453-458 (2013); Brechot, P. P. & Benali, N. L. Cancer Letters 253, 180-204 (2007)). CTCs are rare in the bloodstream—in some cases, only a few CTCs can be identified in a population of 10⁹ blood cells. The effective isolation of CTCs from whole blood is challenging due to the lack of a capturing surface with strong CTC-binding affinity and low off-target binding, which would enable detection of CTCs from the whole blood at a lower detection limit and early diagnosis of cancer. Therefore, the development of effective affinity-based isolation surfaces for CTCs is imperative. Not only do these surfaces need to exhibit both high binding affinity towards CTCs, but also anti-fouling properties against white blood cells (WBCs) (Wu, L. et al. Small Methods 3, 1800544 (2019). Song, Y. et al. Chem. Sci., 8, 1736-1751 (2017). Wu, L. et al. Chem. Rev. 121, 12035-12105 (2021)). As disclosed herein, the inventors have determined compositions and methods that address this need.

### SUMMARY

The present disclosure in the first aspect provides a modified phage comprising on its surface an aptamer that specifically binds to a cancer-related antigen. In some embodiments, the aptamer comprises or consists of the polynucleotide sequence set forth in SEQ ID NO:1. In some embodiments, the aptamer is covalently linked to a first capsid protein of the modified phage. In some embodiments, the modified phage is immobilized on a solid substrate via a second capsid protein of the modified phage. In some embodiments, the modified phage is an M13 phage. In some embodiments, the first capsid protein is pIII, pVI, pVII, pVIII, or pVIX, for example, pVIII. In some embodiments, the covalent link between the aptamer and the first capsid protein comprises an azide linker. In some embodiments, the second capsid protein is pIII, pVI, pVII, pVIII, or pVIX, for example, pIII. In some embodiments, the solid substrate is a magnetic bead.

In a related aspect, the present invention provides a composition that comprises the modified phage of this invention as described above and herein plus one or more pharmaceutically acceptable excipients.

In a second aspect, the present invention provides a method for isolating cancer cells. The method includes these steps: (a) contacting cancer cells with an effective amount of the modified phage of this invention as described above or herein or a composition comprising an effective quantity of the modified phage; and (b) providing conditions that allow specific binding of the modified phage to the cancer cells (e.g., via the aptamer binding to the cancer-related cell surface antigen), thereby isolating the cancer cells by removing the modified phage. In some embodiments, the method further includes, after step (b), a step of releasing cancer cells that are bound to the modified phage. In some embodiments, the method further includes a step of identifying cancer cell subtype, for example, after the cancer cells are released from the modified phage. In some embodiments, the method further comprising a treatment step of administering treatment to a patient, from whom the cancer cells were originally obtained, based on the characteristics of the identified cancer cell subtype. In some embodiments, the cancer cells are breast cancer cells, liver cancer cells, lung cancer cells, prostate cancer cells, colon and rectal cancer cells, stomach cancer cells, pancreatic cancer cells, esophageal cancer cells, kidney cancer cells, bladder cancer cells, brain cancer cells, or ovarian cancer cells.

In a third aspect, the present invention provides a kit for isolating cancer cells. The kit comprising: (a) the modified phage of this invention as described above or herein or a composition containing the modified phage; and (b) an instruction manual for a user on how to use the kit. In some embodiments, the kit further includes a magnet.

### BRIEF DESCRIPTION OF THE DRAWINGS

FIGS. 1A-1C show a schematic of the fabrication of the virus nanofiber-based deformable surface for highly efficient circulating tumor cell (CTC) isolation from whole blood. FIG. 1A shows steps in the engineering of the M13 virus nanofibers. FIG. 1B shows breast cancer CTC capture and profiling using the engineered M13 virus nanofibers and cancer subtyping based on the captured CTCs. ER=estrogen receptor protein. FIG. 1C shows that exemplary engineered M13 virus nanofibers are capable of increased CTC capture and reduced white blood cell (WBC) adsorption.

FIGS. 2A-2C show characterization of aptamer-displaying, flexible M13 nanofibers on magnetic beads (A-f-M13-MB). FIG. 2A, left panel, shows an illustration of the specific interaction between Ni-IDA on microbeads (MBs) and 6His tag on pIII of phage. FIG. 2A, right panel, shows transmission electron microscope (TEM) images of exemplary M13 nanofibers (indicated by arrows)) that were anchored on MBs in an end-on manner. FIG. 2B shows binding efficiency of exemplary of 6His-M13 on Ni-IDA MBs compared to wild type (WT) M13. The results indicate successful construction of 6His-M13 (n=3 samples, mean±s.d.). FIG. 2C shows fluorescence microscopic images of FAM-A-f-M13(MB) (upper panel) and control (lower panel). Aptamer was labeled with FAM. Thus, those MBs anchored with A-f-M13 emitted green fluorescence whereas the control MBs that were anchored with WT-M13 did not emit fluorescence. Scale bar: 20 μm. WT-M13: wild type M13 phage; 6His-M13: M13 with 6His tag displayed on pIII; N3-M13: 6His-M13 with N3-PEG decorated on pVIII; Apt-M13: N3-M13 with aptamer “clicked” onto pVIII.

FIGS. 3A-3H show data related to the mechanical properties of the M13 phage. FIG. 3A relates to M13 stiffness and FIG. 3B relates to M13 Young's modulus, both of which were measured by atomic force microscopy (AFM) under Hertzian mode (n=10 samples, mean±s.d.). Samples measured were untreated M13, EtOH-treated M13 and PFA-treated M13. FIG. 3C shows an AFM image of untreated M13. FIG. 3D shows an AFM image of EtOH-treated M13. FIG. 3E shows an AFM image of PFA-treated M13. The loading amounts of the three types of M13 phages in FIGS. 3C-3E were identical (109 pfu). FIG. 3F shows numerical simulation results for untreated M13. FIG. 3G shows numerical simulation results for EtOH-treated M13. FIG. 3H shows numerical simulation results for PFA-treated M13. The simulation results in FIGS. 3F-3H were under a flow field, the arrows indicate the flow direction, and the subjected flow speed was 2 cm/s. EtOH-treated M13: M13 phage treated with 100% ethanol (EtOH); PFA-treated M13: M13 phage treated with 4% paraformaldehyde (PFA).

FIGS. 4A-4K show that flexible M13 enhanced the capture of CTCs. FIG. 4A, from left to right, shows the dissociation constants (K_{d}) of A-f-M13-MB, A-r-M13-MB, A-MB and free aptamer. The K_{d} value reflects the affinity between CTCs and affinity ligands, with a lower K_{d} corresponding to a higher CTC binding affinity. FIG. 4B shows an illustration of the molecular matching between VNTR repeats in MUC1 and aptamers on Apt-M13, sub-micron-scale matching between MUC1 (200-500 nm in size) and M13 nanofibers (880 nm long), and micron-scale matching between CTCs and M13 nanofiber-anchored topological interfaces. FIGS. 4C-4E show scanning electron microscopy (SEM) images of MCF-7 cells with A-f-M13 (“Flexible M13;” FIG. 4C), A-r-M13 (“Rigid M13;” FIG. 4D), or aptamer (“Apt;” FIG. 4E). FIG. 4F shows percentages of cells that remain on the slide under different relative centrifugal forces (n=3 samples, mean±s.d.). FIG. 4G shows detachment forces required for separating MCF-7 cells from the surface of different slides. The detachment forces were calculated from a centrifugation-based cell adhesion assay. The calculated detachment forces indicated that the CTC binding force (MCF-7 as CTC model) was greatest for flexible M13. Rigid M13 had less calculated detachment force while aptamer had the last calculated detachment force. FIG. 4H shows representative final snapshots of different MB surfaces interacting with CTCs in the simulations. The arrows indicate the aptamer-receptor binding sites. FIG. 4I shows radial distribution function (RDF) of aptamer-specific-bead/receptor-specific-bead pairs in the three cases. RDF indicates the relative density of aptamers around receptors, which was decreased in the order of A-f-M13-MB>A-r-M13-MB>A-MB. FIG. 4J shows total energy of receptor-aptamer interaction was increased in the order of A-f-M13-MB<A-r-M13-MB<A-MB, as more contacts between the specific beads constituting the aptamers and the receptors resulted in lower total energy of the receptor-aptamer interaction (n=101 tests). Unpaired two-sided Student's t-test. The central dot is the median; box bounds are 25^{th} and 75^{th} percentiles, upper and lower limits of whiskers are 1.5× interquartile ranges. Values outside of the upper and lower limits are defined as outliers. *p<0.05, **p<0.01, ***p<0.001, ****p<0.0001. FIG. 4K shows root mean square fluctuation (RMSF) of the aptamer and/or the M13 of the three MBs before and after the CTC adsorption (n=10 tests, mean±s.d.). A-MB: aptamer-modified-magnetic beads; A-f-M13-MB: Ni-IDA-MBs anchored with aptamer-modified-flexible M13; A-r-M13-MB: Ni-IDA-MBs anchored with aptamer-modified-rigid M13 (PFA-treated M13 as a rigid M13 model); Apt: aptamer.

FIGS. 5A-5G show that flexible M13 reduced non-specific absorption of WBCs. FIG. 5A shows the number of WBCs adsorbed on the A-f-M13-MB, A-r-M13-MB and A-MB after 30-min incubation (n=3 samples, mean±s.d.). FIG. 5B shows percentages of residual WBCs on the slide under different relative centrifugal forces (n=3 samples, mean±s.d.). FIG. 5C shows the detachment forces required for separating WBCs from the surface of different slides calculated from the centrifugation-based cell adhesion assay indicated that the WBC binding force (Ramos cell as a model) was increased in the order of flexible M13<rigid M13<aptamer. FIG. 5D shows representative final snapshots of different MB surfaces interacting with WBCs in the simulations. The results indicated that WBCs could be adsorbed onto all three MB surfaces. FIG. 5E shows radial distribution function (RDF) of aptamer-specific-bead/receptor-specific-bead pairs in the three cases. FIG. 5F shows total energy of receptor-aptamer interaction in three cases (n=101 tests). Unpaired two-sided Student's t-test. The central dot is the median; box bounds are 25^{th} and 75^{th} percentiles, upper and lower limits of whiskers are 1.5× interquartile ranges. Values outside of the upper and lower limits are defined as outliers. *p<0.05, **p<0.01, ***p<0.001, ****p<0.0001. Results from FIGS. 5E and 5F revealed the adsorption of WBCs on A-MB was the most energy-favorable, compared to the reduced adsorption of WBCs on A-f-M13-MB and A-r-M13-MB. (n=101 tests) FIG. 5G shows the root mean square fluctuation (RMSF) of the aptamer and/or the M13 of the three MBs before and after the WBC adsorption, indicating that the adsorption of WBCs onto the A-f-M13-MB surface is the least entropy-favorable (n=10 tests, mean±s.d.). A-MB: aptamer-modified-magnetic beads; A-f-M13-MB: Ni-IDA-MBs anchored with aptamer-modified-flexible M13; A-r-M13-MB: Ni-IDA-MBs anchored with aptamer-modified-rigid M13 (PFA-treated M13 as a rigid M13 model); Apt: aptamer.

FIGS. 6A-6F show capture and release performance of A-f-M13-MB. FIG. 6A shows capture efficiency of target cancer cells (MCF-7, A549, HepG2, and SK-Hep-1) using A-f-M13-MB and A-MB in buffer and blood, respectively (n=3 samples, mean±s.d.). The results indicated that A-f-M13-MB can effectively capture the MUC-1 positive cancer cells (MCF-7, A549), but barely adsorbed negative cells (HepG2, SK-Hep-1) both in buffer and whole blood. FIG. 6B shows capture and release performance of A-f-M13-MB, A-r-M13-MB and A-MB towards MCF-7 cells (n=3 samples, mean±s.d.). FIG. 6C shows depletion index value of WBCs with A-f-M13-MB, A-r-M13-MB and A-MB (n=3 samples, mean±s.d.). The depletion index value reflects the purity of CTCs after capture and release, with a higher log₁₀-depletion index value indicating a higher CTC purity. The results revealed a higher CTC purity after A-f-M13-MB capture compared to other two MBs, as well as an improved CTC purity after CTC release. FIG. 6D shows viability of the isolated MCF-7 cells calculated from PI/AO staining results (n=3 samples, mean±s.d.). FIG. 6E shows representative fluorescence microscope images of the isolated live cells and dead cells. The released CTCs were stained by PI/AO, and the live cells emitted green fluorescence and dead cells (indicated by arrows) emitted red fluorescence. Scale bar: 100 μm. Insert is the enlarged dead cells. FIG. 6F shows bright-filed images of the released cells (top) and the cells after being further cultured for 36 h (bottom). The images revealed the released cells were well alive and could be adhered on the well surface 36 h after in vitro cultivation. A-MB: aptamer-modified-magnetic beads; A-f-M13-MB: Ni-IDA-MBs anchored with aptamer-modified-flexible M13; A-r-M13-MB: Ni-IDA-MBs anchored with aptamer-modified-rigid M13 (PFA-treated M13 as a rigid M13 model).

FIGS. 7A-7F show CTC isolation and diagnosis performance in clinical applications. FIG. 7A shows the capture efficiency of different breast cell lines indicated that the CTC affinity interfaces could selectively capture several subtypes of breast cancer cells but not normal breast mammary epithelial cells (n=3 samples, mean±s.d.). FIG. 7B shows the difference in the number of CTCs in 1 mL blood samples from healthy volunteers (n=10), benign patients (n=34), non-metastatic breast cancer (BC) patients (n=32), and metastatic BC patients (n=24). Unpaired two-sided Student's t-test. The central dot is the median; box bounds are 25^{th} and 75^{th} percentiles, upper and lower limits of whiskers are 1.5× interquartile ranges. Values outside of the upper and lower limits are defined as outliers. *p<0.05, **p<0.01, ***p<0.001, ****p<0.0001. FIG. 7C shows ROC analysis of CTC numbers between the cancer patient groups and healthy/benign groups. The receiver operating characteristic (ROC) curve showed good diagnostic performance (AUC=0.991) in differentiating cancer patient groups and healthy/benign groups. FIG. 7D shows immunofluorescence staining (DAPI/ER/HER2/CD45) of CTCs isolated from patients. Scale bar: 10 μm. CD45 was the biomarker for WBC and CK was the biomarker for CTC, with DAPI⁺/CK⁺/CD45⁻ cells recognized as CTCs and DAPI⁺/CK⁻/CD45⁺ cells identified as WBCs. ER and HER2 were used for the profiling of three subtypes of BC CTCs, including luminal (HER2^{+or -}/ER⁺), HER2-positive (HER2⁺/ER⁻) and triple negative breast cancer (TNBC, belongs to basal-like subtype) (HER2⁻/ER⁻). 51 out of 56 molecular profiling results were in accordance with clinical diagnostic results. FIG. 7E shows a confusion matrix showing the subtyping accuracy of luminal, HER2, and TNBC by molecular profiling of CTCs. FIG. 7F shows a comparison of the CTC numbers isolated by A-f-M13-MB and CellSearch® or SE iFISH® in blood revealed a higher CTC capture ability and a better isolation performance of A-f-M13-MB in clinical applications. A-f-M13-MB: Ni-IDA-MBs anchored with aptamer-modified-flexible M13;

FIGS. 8A-8D show the capture and identification of E-type/M-type CTC mixtures by A-f-M13-MB loaded with Y-shaped DNA assembly. FIG. 8A shows an illustration of the construction of Y-shaped DNA assembly. The sequence of anti-vimentine aptamer and anti-EpCAM aptamer were incorporated into scaffold DNA Y₁ and Y ₂, respectively. The three DNA scaffolds Y₁, Y₂ and Y₃ were partially hybridized with each other, thus forming the Y-shaped DNA assembly. FIG. 8B shows that electrophoresis analysis indicates the successful formation of the Y-shaped DNA assembly. Lane 1: marker; lane 2: Y ₁; lane 3: Y ₂; lane 4: Y₃; lane 5: Y ₁₊₃; lane 6: Y ₁₊₂₊₃. FIG. 8C shows the capture efficiency of E-type CTC and M-type CTC by capturing the CTC mixtures by A-f-M13-MB loaded with the Y-shaped DNA assemble (n=3 samples). Error bars represent the mean±s.d. The ratio of E-type: M-Type=9:1, 3:1, 1:1, 1:3 and 1:9. (d) Representative fluorescence microscope image of the isolated CTC mixture. The ratio of E-type: M-Type=1:1, the E-type CTCs were immunostained by Anti-E Cadherin antibody to emit green fluorescence, whereas the M-type CTCs were labeled with red fluorescence by Anti-N Cadherin antibody. Scale bar: 50 μm. Inserted are enlarged images of E-type CTC and M-type CTC. Scale bar: 10 μm. The representative images in FIG. 8D are shown from three independent repeats.

### DETAILED DESCRIPTION

### I. Definitions

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used herein, the term “aptamer” refers to a synthetic single-stranded deoxyribonucleic acid (ssDNA) or a single-stranded ribonucleic acid (ssRNA) molecule. An aptamer can form secondary and tertiary structures, such as helices, steps, loops, hairpins, and quadruplexes, that enable the specific binding of a target, such as a protein, a peptide, a sugar (e.g., a monosaccharide or a polysaccharide), a lipid, a small molecule (e.g., less than 1,500 daltons), a polynucleotide, a cell, a tissue, or a microorganism. An aptamer can be raised against any target of interest and can bind to the target with an affinity that is similar or higher than an antibody, and in some cases, demonstrating a dissociation constant (K_{d}) that ranges from low nanomolar to high picomolar. In general, an aptamer is smaller than an antibody, is non-immunogenic, and are amendable to chemical modifications. Aptamers can range in size from 20-100 nucleotides in length. In addition to high-affinity binding to a target, an aptamer can also bind with high specificity, thereby discriminating between a target protein and off-target proteins. Aptamers are discussed in detail in, e.g., Ni, X et al. Current Medicinal Chemistry vol. 18,27 (2011): 4206-14; and Nimjee, Shahid M et al. Annual Review of Pharmacology and Toxicology vol. 57 (2017): 61-79.

As used herein, the term “bacteriophage” or “phage” refers to a filamentous bacteriophage within the genus Inovirus, for example, the filamentous bacteriophage is an fd, fl, or M13 bacteriophage, typically grouped together and referred to as a “Ff” bacteriophage. Ff bacteriophage are human-friendly viruses that exclusively infect bacteria, often used to serve as a natural and non-toxic bionanofiber utilized in various biomedical applications in research and clinical context. The phage particle comprises a single-stranded DNA enclosed within a tubular capsid composed of five genetically modifiable coat proteins (including pIII and pVI at one end, pVII and pIX at the other end, and pVIII along the sidewall). The sequences of the coat proteins of the filamentous bacteriophages are well known to one of ordinary skill in the art (see, e.g., Kay, B. K., Winter, J. & McCafferty, J., eds. (1996). Phage display of peptides and proteins: a laboratory manual. Academic Press, Inc., San Diego). The coat proteins of Ff phage offer a versatile platform for modifying their genetic codes for the display of specific peptides for the purpose of, e.g., tumor targeting and nanozyme binding.

As used herein, the term “surface” as it relates to a phage or a phage particle refers to the part of a phage/phage particle that is in contact with the medium in which the phage/phage particle is contained. The surface of the phage/phage particle is determined by the coat protein assembly (the assembled members of the protein coat of the particle), including the side walls and rods of the phage/phage particle.

As used herein, the term “cancer” encompasses various malignant neoplasms characterized by the proliferation of anaplastic cells that tend to invade surrounding tissue and metastasize to new body sites. Non-limiting examples of different types of cancer suitable for treatment using the compositions and methods of the present invention include breast cancer, colorectal cancer, colon cancer, anal cancer, liver cancer, ovarian cancer, lung cancer, bladder cancer, thyroid cancer, pleural cancer, pancreatic cancer, cervical cancer, prostate cancer, testicular cancer, bile duct cancer, gastrointestinal carcinoid tumors, esophageal cancer, gall bladder cancer, rectal cancer, appendix cancer, small intestine cancer, stomach (gastric) cancer, renal cancer (e.g., renal cell carcinoma), cancer of the central nervous system, skin cancer, oral squamous cell carcinoma, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, the term “gene” means the segment of DNA involved in producing a polypeptide chain. It may include regions preceding and following the coding region (leader and trailer) as well as intervening sequences (introns) between individual coding segments (exons).

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. As used herein, the term “amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the terms “polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, the phrase “specifically binds,” when used in the context of describing a binding relationship of a particular molecule to a protein or peptide, refers to a binding reaction that is determinative of the presence of the protein in a heterogeneous population of proteins and other biologics. Thus, under designated binding assay conditions, the specified binding agent (e.g., an antibody) binds to a particular protein at least two times the background and does not substantially bind in a significant amount to other proteins present in the sample. Specific binding of an antibody under such conditions may require an antibody that is selected for its specificity for a particular protein or a protein but not its similar “sister” proteins. A variety of immunoassay formats may be used to select antibodies specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically, a specific or selective binding reaction will be at least twice background signal or noise and more typically more than 10, 20, 50, or up to 100 times background. On the other hand, the term “specifically bind” when used in the context of referring to a polynucleotide sequence forming a double-stranded complex with another polynucleotide sequence describes “polynucleotide hybridization” based on the Watson-Crick base-pairing, as provided in the definition for the term “polynucleotide hybridization method.”

As used herein, the term “inhibiting” or “inhibition,” refers to any detectable negative effect on a target biological process, such as protein-protein specific binding or interaction, the biological activity of a target protein, RNA/protein expression of a target gene, cellular signal transduction, cell proliferation, presence/level of an organism especially a micro-organism, any measurable biomarker, bio-parameter, or symptom in a subject, and the like. Typically, an inhibition is reflected in a decrease of at least 10%, 20%, 30%, 40%, 50%, 60%, 70%, 80%, 90% or greater in the target process (e.g., inhibition of a-synuclein aggregation by a protein that was cyclized using an ncAA), or any one of the downstream parameters mentioned above, when compared to a control. “Inhibition” further includes a 100% reduction, i.e., a complete elimination, prevention, or abolition of a target biological process or signal or disease/symptom. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal or disease/symptom. On the other hand, terms such as “activate,” “activating,” “activation,” “increase,” “increasing,” “promote,” “promoting,” “enhance,” “enhancing,” or “enhancement” are used in this disclosure to encompass positive changes at different levels (e.g., at least about 5%, 10%, 20%, 30%, 40%, 50%, 60%, 70%, 80%, 90%, 100%, 200%, or greater such as 3, 5, 8, 10, 20-fold increase compared to a control level in a target process, signal, or symptom/disease incidence.

As used herein, the term an “increase” or a “decrease” refers to a detectable positive or negative change in quantity from a comparison control, e.g., an established standard control (such as an average level of-synuclein aggregation as measured by the Thioflavin T assay). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” as well as terms indicating an action to cause such changes or differences, such as “increase,” “promote,” “enhance,” “decrease,” “inhibit,” and “suppress,” are used in this application in the same fashion as described above. In contrast, the term “substantially the same” or “substantially lack of change” indicates little to no change in quantity from the standard control value, typically within +10% of the standard control, or within +5%, 2%, or even less variation from the standard control.

As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or slowing disease progression, delaying of onset of symptoms, or eliminating or reducing side-effects caused by such disease or condition. A preventive measure in this context and its variations do not require 100% elimination of the occurrence of an event; rather, they refer to a suppression or reduction in the likelihood or severity of such occurrence or a delay in such occurrence.

As used herein, the term “subject,” or “subject in need of treatment,” as used herein, refers to an individual who seeks medical attention due to risk of, or actual sufferance from, a condition involving an undesirable or abnormal, excessive cellular proliferation, for example, solid tumor such as breast cancer. The term subject can include both animals, especially mammals or primates, and humans. Subjects or individuals in need of treatment include those that demonstrate symptoms of undesirable or inappropriate cell proliferation such as tumor and especially malignant tumor/cancer or are at risk of later developing these conditions and/or related symptoms.

As used herein, the term “effective amount,” as used herein, refers to an amount that is sufficient to produce an intended effect for which a substance is administered. The effect may include a desirable change in a biological process (e.g., suppressed cancer cell proliferation) as well as the prevention, correction, or inhibition of progression of the symptoms of a disease or condition and related complications to any detectable extent. The exact amount “effective” for achieving a desired effect will depend on the nature of the therapeutic agent, the manner of administration, and the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

As used in herein, the singular forms “a”, “an” and “the” include plural referents unless the content clearly dictates otherwise. Thus, for example, reference to “an ncAA” optionally includes a combination of two or more such molecules, and the like.

As used herein, the term “about,” when modifying any amount, refers to the variation in that amount typically encountered by one of skill in the art. For example, the term “about” refers to the normal variation encountered in measurements for a given analytical technique, both within and between batches or samples. Thus, the term about can include variation of +/−¹-10% of the measured value, such as +/−1%, 2%, 3%, 4%, 5%, 6%, 7%, 8%, 9%, or 10% variation of the measured value. The amounts disclosed herein include equivalents to those amounts, including amounts modified or not modified by the term “about.”

As used herein, a “pharmaceutically acceptable” or “pharmacologically acceptable” excipient is a substance that is not biologically harmful or otherwise undesirable, i.e., the excipient may be administered to an individual along with a bioactive agent without causing any undesirable biological effects. Neither would the excipient interact in a deleterious manner with any of the components of the composition in which it is contained.

As used herein, the term “excipient” refers to any essentially accessory substance that may be present in the finished dosage form of the composition of this invention. For example, the term “excipient” includes vehicles, binders, disintegrants, fillers (diluents), lubricants, glidants (flow enhancers), compression aids, colors, sweeteners, preservatives, suspending/dispersing agents, film formers/coatings, flavors and printing inks.

As used herein, the term “consisting essentially of,” when used in the context of describing a composition containing an active ingredient or multiple active ingredients, refer to the fact that the composition does not contain other ingredients possessing any similar or relevant biological activity of the active ingredient(s) or capable of enhancing or suppressing the activity, whereas one or more inactive ingredients such as physiological or pharmaceutically acceptable excipients may be present in the composition. For example, a composition consisting essentially of active agents (for instance, the modified phage of this invention) effective for treating cancer in a subject is a composition that does not contain any other agents that may have any detectable positive or negative effect on the same target process (e.g., anti-cancer efficacy) or that may increase or decrease to any measurable extent of the disease severity or outcome among the receiving subjects.

Unless otherwise defined, all technical terms used herein have the same meaning as commonly understood by one of ordinary skill in the art to which this disclosure belongs.

### II. Introduction

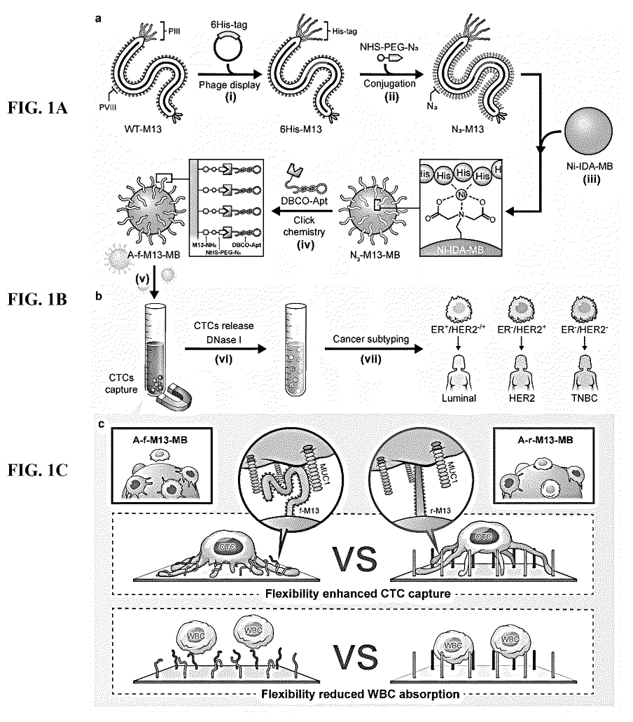

Disclosed herein are compositions and methods for isolating circulating tumor cells (CTCs) from a biological sample. The compositions comprise a modified phage, including a modified M13 bacteriophage (also referred to herein as “phage”), that has been modified to comprise at least one aptamer that binds specifically to a cancer-related antigen (e.g., a cancer cell surface antigen), and has been immobilized on a solid substitute. The mechanical and structural attributes of the modified phage are employed in the efficient isolation of CTCs. See FIGS. 1A-1C. The modified phage comprises a deformable surface that (1) allows the phage maximum flexibility for binding to the antigens on CTCs, and (2) entropically discourages non-specific adsorption white blood cells (WBCs) on the phage's surface (also referred to as “fouling”).

Affinity-based surface bioassays are useful in clinical diagnostics, environmental monitoring, and drug screening. The initial phase of these assays, such as enzyme-linked immunosorbent assays (ELISAs) and immunomagnetic isolation, hinges on the interaction between ligands and receptors, which dictate the assay's affinity. Considerable efforts have been directed towards augmenting binding affinity by tailoring parameters such as ligand type, density, distribution, and conformation (Wu, L. et al. J. Am. Chem. Soc. 142, 4800-4806 (2020); Jeong, W. J. et al. Adv. Sci. 9, 2103098 (2022); Guo, L. et al. Angew. Chem. Int. Ed. 61, e202117168 (2022)). Particularly noteworthy is the challenge posed by complex biological samples containing various matrix components (e.g., blood), where non-specific adsorption of non-target cells or biomolecules significantly undermines assay performance. For example, a patient blood sample may contain low numbers of circulating tumor cells (CTCs) in a complex milieu that includes plasma, red blood cells, white blood cells (also referred to as lymphocytes or white blood cells (WBCs)), and platelets. Non-target cells or biomolecules can occupy active target-binding sites or obstruct on-surface signal transduction of the bioassays. Consequently, a pressing need has emerged for the advancement of surface-shielding strategies (Sabate del Rio, J. et al. Nat. Nanotechnol. 14, 1143-1149 (2019); Banerjee, I. et al. Organisms. Adv. Mater. 23, 690-718 (2011); Jiang, C. et al. Chem. Rev. 120, 3852-3889 (2020)). The application of surface coatings composed of anti-fouling polymers like poly(ethylene glycol) (PEG) or zwitterionic peptides has demonstrated efficacy in reducing non-specific adsorption (Yuan, Z. et al. Angew. Chem. Int. Ed. 59, 22378-22381 (2020); Li, C., et al. Adv. Mater. 31, 1903973 (2019); Maan, A. M. C. et al. Adv. Funct. Mater. 30, 2000936 (2020)). However, the simultaneous presence of anti-fouling polymers and affinity ligands on surfaces unavoidably leads to mutual interference, potentially compromising target-binding affinity or anti-fouling capabilities (Nowinski, A. K. et al. J. Am. Chem. Soc. 134, 6000-6005 (2012); Song, Z. et al. Anal. Chem. 92, 5795-5802 (2020); Tian, Y. et al. J. Am. Chem. Soc. 144, 18419-18428 (2022)). As disclosed herein, the inventors have determined modified phage compositions and related methods with high sensitivity for cancer-related antigens and superior anti-fouling properties.

The M13 phage has a nanofiber-like structure that is 880 nm in length and 6 nm in diameter. Its single-stranded DNA genome resides within a coat comprised of five distinct capsid proteins. Among these, a major capsid protein, pVIII (approximately 2700 copies), is helically arranged along the length of the phage, while four minor capsid proteins, pIII, pVI, pVII, and pIX (5 copies each); cap both ends of the phage, with two of these proteins situated at a single tip (Liu, X. Adv. Mater. 34, 2201210 (2022); Zeng, Y. et al. Angew. Chem. Int. Ed. 61, e202210121 (2022)). Notably, the surface of M13 nanofibers can be tailored through genetic engineering or chemical modification. This versatility enables the orthogonal functionalization of different capsid proteins, yielding desired functionalities—a unique attribute when compared to other artificial analogs. Consequently, the assembly of M13 into scaffolds has yielded remarkable successes in diverse applications, including tissue regeneration, therapy, sensing, and even energy harvesting. These applications are discussed in detail, for example, in Chang, C. et al. Materials Today Bio 20, 100612 (2023); Cao, B. et al. Advanced Drug Delivery Reviews 145, 73-95 (2019); Zhou, N. et al. Adv. Mater. 31, 1905577 (2019); Tsedev, U. et al. ACS Nano. 16, 11676-11691 (2022); Van Nieuwenhuyse, B. et al. Nature Communications 13, 5725 (2022); Peng, H., et al. Proc. Natl. Acad. Sci. U.S.A 117, 1951-1961 (2022); Mao, C. et al. Angew. Chem. Int. Ed. 48, 6790-6810 (2009); Oh, J. W. et al. Nat. Commun. 5, 3043 (2014); and Lee, B. Y. et al. Nature Nanotech. 7, 351-356 (2012).

It was recently discovered that the M13 phage is capable of accelerating mass transport at the liquid-solid interface due to its sway motion (Cao, Y. et al. Small 18, 2203962 (2022)). While most studies focus on the biochemical traits of M13 phage, scant attention has been directed towards the potential impact of its physical or mechanical properties on cellular interactions. The mechanical properties of the phage are relevant to the isolation of circulating tumor cells (CTCs). As disclosed herein, the inventors leverage these properties of filamentous phage in constructing beads that can tightly capture CTCs.

The phage, e.g., M13 phage, bears many copies of the pVIII protein on the sidewall of phage, which provides ample reactive sites for presenting numerous affinity ligands. Furthermore, the phage's inherent flexibility allows it to twist freely. Thus, each ligand's conformation is adjustable and allows for optimal alignment with the distribution pattern of target antigens on the CTC surface (FIG. 1C). This adaptive accommodation significantly enhances the multivalent binding interaction between the M13 phage and CTCs, consequently enabling highly efficient CTC capture. Moreover, owing to its low stiffness and Young's modulus—a quantification of its flexibility—the M13 phage possesses deformability within flowing solutions (Luo, D. et al. J. Am. Chem. Soc. 140, 14211-14216 (2018)). This property results in a heightened binding free energy that counters the non-specific adsorption of WBCs. This entropy-driven effect curbs the background signal from WBCs during CTC capture. Noteworthy is the fact that this phage-based physical shielding strategy for CTC capture obviates the need for additional coatings of anti-fouling polymers. This advantageous feature permits the optimal functionalization of the phage's surface with affinity ligands, thereby augmenting target capture capabilities. Through this physical shielding approach, the phage's deformable surface structure offers a promising avenue for constructing efficient and anti-fouling surface bioassays.

### III. Modified Phage

Disclosed herein are modified phages comprising aptamers. Each modified phage comprises one or more copies of an aptamer that binds specifically to a cancer-related antigen. Typically, the cancer-related antigen is pre-selected and the modified phage comprises an aptamer that binds only to that pre-selected antigen.

Filamentous bacteriophages, including the M13 phage, are well-studied and the surface of these phages can be readily modified via genetic manipulation or chemical conjugation to display functional groups, nucleic acids, peptides, or proteins. In general, functional groups, nucleic acids, peptides, or proteins can be either (1) expressed as fusions with a phage capsid protein, or (2) covalently linked to a phage capsid protein.

Filamentous bacteriophages, including M13 phages, self-assemble into a filamentous structure that provides a large and flexible surface area that can be decorated with an aptamer for interacting with target a cancer-related antigen. The ability to precisely and easily modify surface proteins, as well as display multiple peptides at once, makes this an ideal phage display vector. The phage capsids provide robust protection for viruses in unpredictable human body environments to maintain concentration and stability after administration into a human subject. Being one of the most abundant biological entities, filamentous phage can generate large numbers of progeny phages upon infecting bacteria, making it an economical and accessible source of biomedical material. By virtue of the lysogenic cycle and error-free replication, filamentous phage can be safely used in vivo without damaging mammalian cells. Filamentous phages, e.g., M13 phages, are discussed in detail in, e.g., Frei, J C, and J R Lai. Methods in Enzymology vol. 580 (2016): 45-87; Davydova, Elena K. Biochemistry. Biokhimiia vol. 87, Suppl 1 (2022): S146-S110; and Chang, Cheng et al. Materials Today Bio. vol. 20 100612 (2023).

A filamentous bacteriophage, e.g., M13 phage, comprises pIII, pVI, pVII, pVIII, and pIX capsid proteins. pVIII is the most abundant capsid protein—about 2700 copies of pVIII can be found on the side wall of the phage body (FIG. 1A). In some embodiments, the aptamer is linked to pVIII and the modified phage can comprise a plurality of aptamers (FIG. 1A).

The M13 filamentous phage, for example, comprises five copies of each of the minor capsid proteins pIII, pVI, pVII, and pIX. pIII and pVI are located on one end of the phage's filamentous structure, and pVII and pIX are located on the opposite end (FIG. 1A). In some embodiments, the phage is immobilized on a solid surface via a linker to pIII, pVI, pVII, or pIX. In some embodiments, the phage is immobilized on a solid surface via a linker to pIII (FIG. 1A). In some embodiments, the modified phage is a modified M13 phage.

a. Aptamer Characteristics, Selection, and Modifications

The modified phages disclosed herein each comprise a surface that is decorated with one or more copies of an aptamer (FIG. 1A). The aptamers disclosed herein are synthetic oligonucleotides, e.g., ssDNA or ssRNA molecules, that bind to cancer-related antigens with high specificity and affinity. In this way, the modified phages are useful for binding to cancer cells and/or for isolating cancer cells. Compared to traditional antibodies, aptamers have the advantage of higher stability over a wide range of temperatures, pH, and solvents, making them useful in a wide range of conditions. Furthermore, aptamers are easily modifiable with various functional moieties, and can be synthesized via chemical or enzymatic approaches, earning them the moniker of “chemical antibodies.” In addition, aptamers would not cause unnecessary immunological responses due to the absence of fragment crystallizable (Fc) regions, which would bind with Fc receptors expressed on the surface of immune cells. These properties give aptamers a low immunogenicity and good biocompatibility. The advantages of using aptamers in disease diagnostics and therapeutics are discussed in detail in, for example, Hu, Xueqi et al. Life (Basel, Switzerland) vol. 12,11 1937. 21 Nov. 2022.

In some embodiments, the aptamer comprises at least 20, 30, 40, 50, 60, or 70 nucleotides. In some embodiments, the aptamer is 20-40 nucleotides, 20-50 nucleotides, 20-60 nucleotides, 30-40 nucleotides, 30-50 nucleotides, or 30-60 nucleotides in length.

Aptamer binding is highly dependent on the secondary structure formed by the aptamer oligonucleotide. Both RNA and single stranded DNA (or analog) aptamers are known. See,. e.g. Burke et al. (1996). J. Mol. Biol. 264:650-666; Ellington and Szostak (1990). Nature 346:818-22; Hirao et al. (1998). Mol Divers. 4:75-89; Jaeger et al. (1998). EMBO Journal 17:4535; Kensch et al. (2000). J. Biol. Chem. 275:18271-8; Schneider et al. (1995). Biochemistry 34:9599-9610; and U.S. Pat. Nos. 5,773,598; 6,028,186; 6,110,900; 6,127,119; and 6,171,795.

Aptamers can be designed and optimized for binding to cancer-related antigens. In some embodiments, the aptamers are used to bind to and/or isolate circulating tumor cells (CTCs) from a biological sample, e.g., blood. In general, aptamers can be selected using a biopanning method, for example, a method known as Systematic Evolution of Ligands by Exponential Enrichment (SELEX™). See, e.g., Ellington A.D. and Szostak J. W. Nature. 1990; 346(6287):818-822; and Tuerk C. and Gold L. Science. 1990; 249(4968):505-510; and U.S. Pat. Nos. 5,475,096 and 5,270,163, and PCT Publication No. WO 1991/019813. This method entails iterative screening of high-affinity oligonucleotides against a diverse range of targets, which can include small molecules, proteins, peptides, toxins, cells, and tissues. The classic SELEX™ method starts with a random sequence library of ssDNA or ssRNA that spans 20-100 nucleotides (nt) in length. The randomization of nucleic acid sequences provides a diversity of 4ⁿ, with n corresponding to the number of randomized bases. For example, a 20 nucleotide randomized segment of an aptamer can have 4²⁰ sequence possibilities. While seemingly infinite diversities can be achieved by this method, only diversities of ˜10¹⁶ aptamers can be readily generated and screened. Each random sequence region is flanked by constant sequences required for capture or priming. The initial diverse pool of aptamers is then exposed to a target molecule, with the expectation that a portion of the aptamers can fold in such a way that they will specifically bind to the target molecule. Non-binding aptamers are then washed away, while candidate aptamers with high target binding affinity are enriched at each selection round by PCR amplification (DNA aptamers) or RT-PCR followed by in vitro transcription (RNA aptamers). The enriched pool of aptamers is then exposed to the target again, and the process repeats. During this iterative process, the aptamer pool can also be counter-selected: where the pool is incubated with unwanted targets in order to deplete it of non-specific binders. After multiple rounds of target selection and enrichment, aptamer pools will show increase binding affinity and begin to converge to one or more consensus sequences. Finally, individual aptamer clones can be generated and tested for target binding specificity and affinity. Since its introduction in 1990, approximately 20 variants of original SELEX™ techniques have been developed. See, e.g., Radom, Filip et al. Biotechnology Advances. vol. 31,8 (2013): 1260-74; and Aquino-Jarquin, G. and Toscano-Garibay, J.D. International Journal of Molecular Sciences. vol. 12,12 (2011): 9155-71. For example, the AptaBid method can be used to generate aptamers and finds novel biomarkers in parallel (Berezovski M. V. et al., J. Am. Chem. Soc. 2008; 130(28):9137-9143). A series of counter- and positive-selections are designed to enrich for aptamers which bind biomarkers that are differentially expressed on cells in different states. The candidate aptamers can be used to isolate and identify their binding partner, or biomarker, by mass spectrometry. This is one example where aptamer diversity and library screens can be used to generate new clinical tools or assays for disease detection, management, or therapy.

SELEX™ and related methods rely on, as a starting point, a large library or pool of single stranded oligonucleotides comprising randomized nucleic acid sequences. The oligonucleotides can be modified or unmodified DNA, RNA, or DNA/RNA hybrids. In some examples, the pool comprises 100% random or partially random oligonucleotides. In other examples, the pool comprises random or partially random oligonucleotides containing at least one fixed and/or conserved sequence incorporated within randomized sequence. In other examples, the pool comprises random or partially random oligonucleotides containing at least one fixed and/or conserved sequence at its 5′ and/or 3′ end which may comprise a sequence shared by all the molecules of the oligonucleotide pool. Fixed sequences are sequences such as hybridization sites for PCR primers, promoter sequences for RNA polymerases (e.g., T3, T4, T7, and SP6), restriction sites, or homopolymeric sequences, such as poly A or poly T tracts, catalytic cores, sites for selective binding to affinity columns, and other sequences to facilitate cloning and/or sequencing of an oligonucleotide of interest. Conserved sequences are sequences, other than the previously described fixed sequences, shared by a number of aptamers that bind to the same target. In some cases, the oligonucleotides of the starting pool contain fixed 5′ and 3′ terminal sequences which flank an internal region of 30-50 random nucleotides. The oligonucleotides can be produced using a number of different methods, including chemical synthesis and size selection from randomly cleaved cellular nucleic acids. Sequence variation in test oligonucleotides can also be introduced or increased by mutagenesis before or during the selection/amplification iterations.

The random sequence portion of the oligonucleotide can be of any length and can comprise ribonucleotides and/or deoxyribonucleotides and can include modified or non-natural nucleotides or nucleotide analogs. See, e.g. U.S. Pat. Nos. 5,958,691; 5,660,985; 5,958,691; 5,698,687; 5,817,635; and 5,672,695; and PCT Publication WO 92/07065. Random oligonucleotides can be synthesized from phosphodiester-linked nucleotides using solid phase oligonucleotide synthesis techniques well known in the art. See, e.g., Froehler et al., Nucl. Acid Res. 14:5399-5467 (1986) and Froehler et al., Tet. Lett. 27:5575-5578 (1986). Random oligonucleotides can also be synthesized using solution phase methods such as triester synthesis methods. See, e.g., Sood et al., Nucl. Acid Res. 4:2557 (1977) and Hirose et al., Tet. Lett., 28:2449 (1978). Typical syntheses carried out on automated DNA synthesis equipment yield 10¹⁴-10¹⁶ individual molecules, a number sufficient for most SELEX experiments. Sufficiently large regions of random sequence in the sequence design increases the likelihood that each synthesized molecule is likely to represent a unique sequence.

The starting library of oligonucleotides may be generated by automated chemical synthesis on a DNA synthesizer. To synthesize randomized sequences, mixtures of all four nucleotides are added at each nucleotide addition step during the synthesis process, allowing for random incorporation of nucleotides. As stated above, in some embodiments, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

The starting library of oligonucleotides may be for example, RNA, DNA, or RNA/DNA hybrid. In those instances where an RNA library is to be used as the starting library it is typically generated by transcribing a DNA library in vitro using T7 RNA polymerase or modified T7 RNA polymerases and purified. The library is then mixed with the target under conditions favorable for binding and subjected to step-wise iterations of binding, partitioning and amplification, using the same general selection scheme, to achieve virtually any desired criterion of binding affinity and selectivity. More specifically, starting with a mixture containing the starting pool of nucleic acids, the SELEX™ method includes steps of (a) contacting the mixture with the target under conditions favorable for binding; (b) partitioning unbound nucleic acids from those nucleic acids which have bound specifically to target molecules; (c) dissociating the nucleic acid-target complexes; (d) amplifying the nucleic acids dissociated from the nucleic acid-target complexes to yield a ligand-enriched mixture of nucleic acids; and (e) reiterating the steps of binding, partitioning, dissociating and amplifying through as many cycles as desired to yield highly specific, high affinity nucleic acid ligands to the target molecule. In those instances where RNA aptamers are being selected, the SELEX™ method further comprises the steps of: (i) reverse transcribing the nucleic acids dissociated from the nucleic acid-target complexes before amplification in step (d); and (ii) transcribing the amplified nucleic acids from step (d) before restarting the process.

Within a nucleic acid mixture containing a large number of possible sequences and structures, there is a wide range of binding affinities for a given target. A nucleic acid mixture comprising, for example, a 30 nucleotide randomized segment can have 4³⁰ candidate possibilities. Those which have the higher affinity constants for the target are most likely to bind to the target. After partitioning, dissociation, and amplification steps (i.e., selection), a second nucleic acid mixture is generated, enriched for the higher binding affinity candidates. Additional rounds of selection progressively favor better ligands until the resulting nucleic acid mixture is predominantly composed of only one or a few sequences. These can then be cloned, sequenced, and individually tested for binding affinity as pure ligands or aptamers.

Cycles of selection are repeated until a desired goal is achieved. In the most general case, selection is continued until no significant improvement in binding strength is achieved on repetition of the cycle. Generally, nucleic acid aptamer molecules are selected in a 5 to 20 cycle procedure, although in some cases, more cycles are beneficial. Sequence variation in the aptamer candidates can be introduced or increased by mutagenesis before or during selection/amplification iterations. Enrichment of aptamer candidates may be monitored using restriction fragment length polymorphism (RFLP) and flow cytometry as described in Shigdar S et al. (2013) Cancer Letters 330:84-95.

A variety of nucleic acid primary, secondary, and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX™ procedures with contiguous randomized segments be initiated with nucleic acid sequences containing a randomized segment of between about 20 to about 50 nucleotides and in some embodiments, about 20 to about 35 nucleotides, or about 30 to about 40 nucleotides. For example, an aptamer comprising a 5′-fixed:random:3′-fixed sequence comprises a random sequence of about 30 to about 50 nucleotides.

The binding affinity describes the measure of the strength of the binding or affinity of molecules to each other. Binding affinity of an aptamer disclosed herein with respect to a target and other molecules is defined in terms of a dissociation constant, K_{d}. The dissociation constant can be determined by methods known in the art and can be computed even for complex mixtures by methods such as those, for example, set forth in Caceci, M., et al., Byte (1984) 9:340-362. Examples of measuring dissociation constants are described for example in U.S. Pat. No. 7,602,495, which describes surface plasmon resonance analysis, and in U.S. Pat. No. 6,562,627, and U.S. Application Publication No. 2012/00445849. In another example, the K_{d} is established using a double-filter nitrocellulose filter binding assay such as that disclosed by Wong and Lohman, (1993). Proc. Natl. Acad. Sci. USA 90, 5428-5432. Methods for determining binding affinity of aptamers is also described in for example, Stoltenburg R et al. (2005) Anal Bioanal Chem 383:83-91, Tran D T et al (2010) Molecules 15, 1127-1140, and Cho M. et al. (2013) Proc. Natl. Acad. Sci. USA 110(46):18460-18465.

In some embodiments, the aptamer binds specifically to the cancer-related antigen with a dissociation constant, K_{d}, of at least 0.1 pM, at least 1 pM, at least 10 pM, at least 20 pM, at least 50 pM, at least 100 pM, at least 1 nM, 0.1-1 pM, 0.1-10 pM, 0.1-20 pM, 0.1-50 pM, 0.1-100 pM, 1-100 pM, 10-100 pM, 20-100 pM, 50-100 pM, or 0.1 pM to 1 nM, as determined by fluorescence measurements, for example, by Fluorescence Intensity-Based Assays, which are known in the art as well as described herein. Additional methods for measuring K_{d} include (1) Equilibrium Dialysis, (2) Surface Plasmon Resonance (SPR), (3) Isothermal Titration Calorimetry (ITC), (4) Fluorescence-Based Methods (including Fluorescence Anisotropy (FA)/Fluorescence Polarization (FP), Fluorescence Resonance Energy Transfer (FRET), and Fluorescence Intensity-based assay), (5) Radioactive Ligand Binding Assays, (6) Electrophoretic Mobility Shift Assay (EMSA).

Also disclosed herein are analogs as described herein and/or additional modifications designed to improve one or more characteristics of the aptamers such as protection from nuclease digestion. For example, one potential problem that is often encountered in the use of nucleic acids as a diagnostic and/or therapeutic tool is that an oligonucleotide in its phosphodiester form may be quickly degraded by intracellular and extracellular enzymes (such as endonucleases and exonucleases found in, for example, body fluids in a subject or body fluids contained in a biological sample that was obtained from a subject) before the desired effect is manifest. Resistance to nuclease degradation can be greatly increased by the incorporation of modifying groups, e.g., a fluoro or amino group at the 2′-position, or a methyl group at the 2′-O-position. Aptamer modifications contemplated in the present disclosure include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications to generate aptamers that are resistant to nucleases can also include one or more substitute internucleotide linkages, altered sugars, altered bases, or combinations thereof. Such modifications include 2′-position sugar modifications, 5-position pyrimidine modifications, 8-position purine modifications, modifications at exocyclic amines, substitution of 4-thiouridine, substitution of 5-bromo or 5-iodo-uracil, backbone modifications, phosphorothioate or alkyl phosphate modifications, methylations, and unusual base-pairing combinations such as the isobases isocytidine and isoguanosine; 3′ and 5′ modifications such as capping; conjugation to a high molecular weight, non-immunogenic compound; conjugation to a lipophilic compound; and phosphate backbone modification.

In some embodiments, the non-immunogenic, high molecular weight compound conjugated to the aptamer of the present disclosure is polyalkylene glycol, preferably polyethylene glycol. In some embodiments, the backbone modification comprises incorporation of one or more phosphorothioates into the phosphate backbone. In another example, the aptamer of the present disclosure comprises the incorporation of fewer than 10, fewer than 6, or fewer than 3 phosphorothioates in the phosphate backbone.

Where appropriate, additional modifications may include at least one of the following, 2′-deoxy, 2′-halo (including 2′-fluoro), 2′-amino (preferably not substituted or mono- or disubstituted), 2′-mono-, di- or tri-halomethyl, 2′-O-alkyl, 2′-O-halo-substituted alkyl, 2′-alkyl, azido, phosphorothioate, sulfhydryl, methylphosphonate, fluorescein, rhodamine, pyrene, biotin, xanthine, hypoxanthine, 2,6-diamino purine, 2-hydroxy-6-mercaptopurine and pyrimidine bases substituted at the 6-position with sulfur or 5 position with halo or C15 alkyl groups, abasic linkers, 3′-deoxy-adenosine as well as other available “chain terminator” or “non-extendible” analogs (at the 3′-end of the RNA), or labels such as ³²P, ³³P and the like.

In some embodiments, aptamers are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R is independently H or substituted or unsubstituted alkyl. Linkage groups can be attached to adjacent nucleotides through an —O—, —N—, or —S— linkage. Not all linkages in the oligonucleotide are required to be identical. As used herein, the term phosphorothioate encompasses one or more non-bridging oxygen atoms in a phosphodiester bond replaced by one or more sulfur atoms.

In further embodiments, the aptamers comprise modified sugar groups. For example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat et al., Nucl. Acid Res. 19:733-738 (1991); Cotten et al., Nucl. Acid Res. 19:2629-2635 (1991); and Hobbs et al., Biochemistry 12:5138-5145 (1973).

The SELEX™ method may be used to identify of high-affinity aptamers containing modified nucleotides conferring improved characteristics on the aptamer, such as improved in vivo stability or improved delivery characteristics. Examples of such modifications include chemical substitutions at the ribose and/or phosphate and/or base positions. SELEX™ identified aptamers containing modified nucleotides are described, e.g., in U.S. Pat. No. 5,660,985, which describes aptamers containing nucleotide derivatives chemically modified at the 2′ position of ribose, 5 position of pyrimidines, and 8 position of purines; U.S. Pat. No. 5,756,703, which describes oligonucleotides containing various 2′-modified pyrimidines; and U.S. Pat. No. 5,580,737 which describes highly specific nucleic acid ligands containing one or more nucleotides modified with 2′-amino (2′-NH₂), 2′-fluoro (2′-F), and/or 2′-O-methyl (2′-OMe) substituents.

All of the foregoing can be incorporated into an aptamer using the standard synthesis and/or conjugation techniques disclosed herein and elsewhere, and are readily available to one skilled in the art.

b. Cancer-Related Antigens

The modified phage of the present disclosure comprising an aptamer can bind specifically to a pre-selected antigen of interest, such as a cancer-related cell surface antigen. A cancer-related antigen is typically a protein or other molecule that is associated with a cancer cell and not with a normal cell (also referred to as a healthy or noncancerous cell). In some cases, the cancer-related antigen is on the cancer cell, meaning that it is found on the outside of the cancer cell. In some cases, the cancer-related antigen is a protein and it is expressed on the surface of the cancer cell.

In some cases, cancer-related antigens are useful as possible targets for diagnosing cancer type. For example, a modified phage, e.g., a modified M13 phage, of the present disclosure can be used as a capture agent to bind or immobilize a cancer cell expressing a cancer-related antigen on its surface. In some cases, the cancer-related antigens are useful as possible targets for targeted cancer therapy. For example, a modified phage, e.g., a modified M13 phage, of the present disclosure can be used as a targeting agent to deliver a therapeutic molecule to a cancer cell expressing a cancer-related antigen on its surface. In some cases, cancer-related antigens may be useful in helping the body produce an immune response against cancer cells. For example, a modified phage, e.g., a modified M13 phage, of the present disclosure can bind to a cancer-related antigen on a cancer cell surface, and recruit immune cells to a cancer cell to facilitate killing of the cancer cell.

Non-limiting examples of cancer-related antigens include human mucin-1 (MUC1; UniProt ID: P15941), human epidermal growth factor receptor 2 (HER2; receptor tyrosine-protein kinase erbB-2; UniProt ID: P04626), and human epithelial cell adhesion molecule (EpCAM; UniProt ID: P16422). Further non-limiting examples of cancer-related antigens (e.g., biomarkers) are disclosed in Tables 1-7 of Hu, Xueqi et al. Life (Basel, Switzerland) vol. 12,11 1937. 21 Nov. 2022.

In some embodiments, the modified phage, e.g., modified M13 phage, comprises an aptamer comprising a nucleic acid sequence that binds specifically to human mucin-1 (MUC1; UniProt ID: P15941). In some embodiments, the aptamer comprises a nucleic acid sequence comprising at least 95% sequence identity with any one of SEQ ID NOs:1-4. In some embodiments, the aptamer comprises a nucleic acid sequence as set forth in any one of SEQ ID NOs:1-4. In some embodiments, the aptamer is any one of SEQ ID NOs:1-4. In some embodiments, the aptamer comprises a modification, e.g., a dibenzocyclooctyne (DBCO) label or a fluorescent label, as shown in any one of SEQ ID NO:1-4. DBCO labeling and other methods of labeling used to conjugate an aptamer to the M13 phage are discussed in detail below. Fluorescent labeling and other methods of labeling an aptamer with a detectable label area also discussed in detail below.

In some embodiments, the modified phage, e.g., modified M13 phage, comprises an aptamer comprising a polynucleotide sequence that binds specifically to a cancer-related biomarkers, for example, any one of those named in Table 1 of Kim et al., Molecules 2018, 23, 830, reproduced below:

| | Known Expressing | Therapeutic | | |
|---|---|---|---|---|
| Target | Cancer Type | Application | [symbol] | Reference |
| [symbol] | [symbol] | [symbol] | [symbol] | [symbol] |
| | [symbol] | | [symbol] | [symbol] |
| [symbol] | [symbol] | [symbol] | [symbol] | [symbol] |
| | [symbol] | [symbol] | [symbol] | [symbol] |
| [symbol] | [symbol] | [symbol] | [symbol] | [symbol] |
| | [symbol] | | | |
| [symbol] | [symbol] | [symbol] | [symbol] | [symbol] |
| | [symbol] | [symbol] | [symbol] | [symbol] |
| | | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | | [symbol] | [symbol] | [symbol] |
| [symbol] | [symbol] | [symbol] | [symbol] | [symbol] |
| | [symbol] | [symbol] | | |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | [symbol] | | | |
| [symbol] | [symbol] | [symbol] | [symbol] | [symbol] |
| | [symbol] | | [symbol] | [symbol] |
| | [symbol] | | [symbol] | [symbol] |
| [symbol] | [symbol] | [symbol] | [symbol] | [symbol] |

[symbol]

indicates data missing or illegible when filed

The modified phages disclosed herein have low affinity for cells that have low or non-detectable levels of a cancer-related antigen, e.g., MUC1, HER2, EpCAM, etc. Non-limiting examples of cancer-related antigens are discussed above. In general, these cells are referred to as noncancerous cells. Non-limiting examples of cells that have low or non-detectable levels of cancer-related antigen comprise white blood cells (monocytes, lymphocytes, neutrophils, eosinophils, basophils, and macrophages), red blood cells (erythrocytes), and platelets.

c. Conjugating Aptamers to Phage

A variety of methods can be used to covalently link (or “conjugate”) an aptamer to the surface of a modified phage, e.g., a modified M13 phage, including, for example, click chemistry methods. In general, the term “click chemistry” relates to a set of chemical reactions using readily available starting reagents that are compatible with an aqueous environment or no solvent, and that may require simple separation step(s) to isolate the desired product (e.g., chromatographic methods). Click chemistry methods typically involve reactions that are broad in scope, high yielding, stereospecific, and result in few or no byproducts. Exemplary reactions that follow these criteria include (1) nucleophilic substitutions, (2) additions to C—C multiple bonds (e.g., Michael addition, epoxidation, dihydroxylation, or aziridination), (3) nonaldol-like chemistry (e.g., N-hydroxysuccinimide active ester couplings), and (4) cycloadditions (e.g., Diels-Adler reaction, or Huisgen's cycloaddition). Click chemistry methods are discussed in detail in, e.g., Fantoni, Nicolò Zuin et al. Chemical Reviews. vol. 121,12 (2021): 7122-7154; Kolb, Hartmuth C. et al. Angewandte Chemie (International ed. in English) vol. 40,11 (2001): 2004-2021; Carmody, Caitlin M et al. Bioconjugate Chemistry vol. 32,3 (2021): 466-481; and Marks, Isaac S et al. Bioconjugate Chemistry vol. 22,7 (2011): 1259-63.

In some embodiments, an aptamer is modified to comprise a dibenzocyclooctyne group (also referred to as “DBCO” or “DIBO”), and the DBCO covalently binds to an azide group on the phage (see, e.g., “N₃-M13” in Example 1 below). In this way, the phage can be decorated with one or more aptamers. In some embodiments, a click chemistry method, such as a copper-free click chemistry method is used to perform this covalent linkage reaction, for example, as discussed in Yuan, R. et al. Anal. Chem. 91, 4948-4952 (2019). Briefly, in these embodiments, the aptamer is first modified to comprise a DBCO functional group, and the phage is modified to incorporate an azide group by coupling with an active ester to form an amido bond in sterile PBS (for example, as discussed in Example 1 below). Aptamer decoration of the phage is then achieved via click chemistry between the DBCO-aptamer and the phage (see, e.g., “N₃-M13” in Example 1 below).

Aptamer can be linked to the proteins constituting the surface of the phage by other methods, including: (1) EDC/NHS conjugation (EDC=1-ethyl-3-(3-dimethylaminopropyl) carbodiimide; NHS═N-hydroxysuccinimide): This method uses carbodiimide chemistry to link carboxyl groups modified on apatamer to amine groups on proteins; (2) Maleimide-Thiol Conjugation: Thiol groups introduced into the apatmer and maleimide introduced into the phage protein can react to form stable thioether bonds; (3) Biotin-Streptavidin Interaction: Biotin introduced into apatamer and streptavidin introduced into the phage protein can bind to form a complex.

In some embodiments, the aptamer is covalently linked to pIII, pVI, pVII, pVIII, or pVIX capsid protein of the modified phage, e.g., the modified M13 phage. In some embodiments, the aptamer is covalently linked to pVIII capsid protein. In some embodiments, the aptamer is linked to pVIII capsid protein via a DBCO-azide linkage.

d. Phage Immobilization on a Solid Surface

The modified phages, e.g., modified M13 phages, of the present disclosure, may be immobilized on a solid surface. A modified phage may be linked directly or indirectly to a solid surface or substrate. See, e.g., FIG. 1A. A solid surface or substrate can be any physically separable solid to which a modified phage can be directly or indirectly attached including, but not limited to, surfaces provided by particles such as beads, microarrays and wells, columns, optical fibers, wipes, glass and modified or functionalized glass, quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polypropylene, polyethylene, polybutylene, polyurethanes, Teflon material, etc.), polysaccharides, nylon or nitrocellulose, resins, silica or silica-based materials including silicon and modified silicon, carbon, metals, inorganic glasses, plastics, ceramics, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In addition, as is known the art, the substrate may be coated using passive or chemically-derivatized coatings to provide one or more functional groups on a solid surface. Coating can comprise of any number of materials, including polymers, such as dextrans, acrylamides, gelatins or agarose. Such coatings can facilitate the use of the array with a biological sample. Functional groups can include amine groups, carboxyl groups, thiol groups, biotin, SiO₂, EDTA, and boronic acid functional groups. In some embodiments, the coating is an ion-functionalized coating, such as a nickel- or cobalt-functionalized coating. In some embodiments, a solid surface may comprise one or more members of a specific binding pair. For example, avidin, streptavidin, Neutravidin®, Captavidin™, or biotin may be positioned on a solid surface.

In some embodiments, the modified phage, e.g., modified M13 phage, is linked to a solid surface, for example, the surface of a particle. In some embodiments, a plurality of phages is immobilized on a single particle. Suitable particles may be sized to have at least one dimension, e.g., diameter, of from about 50 nm to about 100 μm. For example, in some embodiments a suitable particle is sized to have at least one dimension of from about 50 nm to about 1 μm, e.g., from about 50 nm to about 500 nm, or from about 50 nm to about 100 nm. In other embodiments, a suitable particle is sized to have at least one dimension of from about 500 nm to about 100 μm, e.g., from about 1 μm to about 100 μm, or from about 50 μm to about 100 μm. Suitable particles may be generally spherical or may have any other suitable shape.

Particles may be made from a variety of suitable materials known in the art. For example, magnetic particles may be utilized in the disclosed methods and compositions. Suitable magnetic particles may include, for example, magnetic beads or other small objects made from a magnetic material such as a ferromagnetic material, a paramagnetic material, or a superparamagnetic material. Magnetic particles may include, e.g., iron oxide (Fe₂O₃ and/or Fe₃O₄). Additional particles of interest may include polymer based particles, e.g., polymer based beads. For example, polystyrene particles may be utilized. In addition, in some embodiments ceramic particles may be utilized. In some embodiments, the particles may include or be coated with a material which facilitates coupling of the particles to aptamers. Examples of coatings include polymer shells, glasses, ceramics, gels, etc. In some embodiments, the coatings include or are themselves coated with a material that facilitates coupling or physical association of the particles with the aptamers. For example, particles with exposed carboxylic acid groups may be used for attachment to aptamers. In some embodiments, the particle is an ion-functionalized particle, such as a nickel- or cobalt-functionalized particle.

A variety of methods can be used to immobilize a modified phage, e.g., modified M13 phage, to a solid surface. In some embodiments, the solid surface comprises an ion-functionalized surface and the phage comprises an affinity tag for the ion-functionalized surface. In some embodiments, the solid surface is an ion-chelated solid surface, e.g., a magnetic bead (or microbead (MB)) or a glass slide. In some embodiments, the ion is a nickel or cobalt. In some embodiments, the phage is genetically modified to express a suitable tag, such as a six-histidine peptide (6His tag). In some embodiments, the phage comprises a six-histidine peptide (6His tag) on a capsid protein of the phage, for example, at the N-terminus of the capsid protein. In some embodiments, the capsid protein is pIII, pVI, pVII, pVIII, or pVIX. In some embodiments, the capsid protein is pIII. See FIG. 1A. Methods of localizing complexes, including phages, using nickel ions, 6His tag, and nitrilotriacetic acid (NTA) are known to one of ordinary skill in the art, for example, as discussed in Alarcó n-Correa, M. et al. ACS Nano. 13, 5810-5815 (2019); and Conti, M. et al. Angew. Chem. Int. Ed. 112, 221-224 (2000). Methods of genetically modifying an M13 phage are known to one of ordinary skill in the art, for example, as discussed in Shen, W. et al. J. Am. Chem. Soc. 131, 6660-6661 (2009).

A modified phage, e.g., modified M13 phage, of the present disclosure can be included in an array for detection and characterization of CTCs. Array surfaces useful may be of any desired shape, form, or size. Non-limiting examples of surfaces include chips, continuous surfaces, curved surfaces, flexible surfaces, films, plates, sheets, or tubes. Surfaces can have areas ranging from approximately a square micron to approximately 500 cm². The area, length, and width of surfaces may be varied according to the requirements of the assay to be performed. Considerations may include, for example, ease of handling, limitations of the material(s) of which the surface is formed, requirements of detection systems, requirements of deposition systems (e.g., arrayers), or the like. In certain embodiments, it is desirable to employ a physical means for separating immobilized phages where each phage binds to a different cancer-related antigen: such physical separation facilitates exposure of different groups or arrays to different solutions of interest. Therefore, in certain embodiments, arrays are situated within microwell plates having any number of wells. In such embodiments, the bottoms of the wells may serve as surfaces for the formation of arrays, or arrays may be formed on other surfaces and then placed into wells.

In addition, a surface-binding peptide may be first screened and identified from a phage-displayed random peptide library. Then the surface-binding peptide can be displayed at the tip of phage. The tip of the phage can lastly bind to the solid surface to achieve end-on immobilization.

### IV. Methods of Using the Modified Phage

a. Detection of Cancer-Related Antigens

Circulating tumor cells (CTCs) are released from primary tumors and transported through the body via blood or lymphatic vessels before settling to form micrometastases under suitable conditions. Accordingly, several studies have identified CTCs as a negative prognostic factor for survival in many types of cancer. CTCs also reflect the current heterogeneity and genetic and biological state of tumors; so, their detection and characterization through liquid biopsy can provide valuable insights into disease progression and patient prognosis as well as inform suitable treatment options. Their extreme rarity, however, requires isolation from large blood volumes at high yield and purity, yet they overlap leukocytes in size and other biophysical properties.

The modified phages of the present disclosure, e.g., modified M13 phages, can be used in vitro for diagnostic and/or detection purposes to determine the presence of cells that express cancer-related antigens on their surface, for example, MUC1-expressing CTCs. The method involves examining a biological sample from a subject, e.g., blood, for the presence of a cancer-related antigen, e.g., MUC1, that are expressed on CTCs. For example, a biological sample can be contacted with a modified phage, e.g., modified M13 phage, of the present disclosure that comprises an aptamer of the present disclosure. The modified phage is analyzed for its ability to specifically bind to a cancer-related antigen, thereby binding to, and in some cases, isolating and/or producing an enriched sample of the cells that express the cancer-related antigen in the biological sample. In some embodiments, phage binding indicates the presence of a cell expressing a cancer-related antigen. In some embodiments, phage binding indicates the level of cancer-related antigen that is present in the biological sample. In some cases, the method can be used to identify the subtype of a cancer cell. In some embodiments, the cancer cells are breast cancer cells, liver cancer cells, lung cancer cells, prostate cancer cells, colon and rectal cancer cells, stomach cancer cells, pancreatic cancer cells, esophageal cancer cells, kidney cancer cells, bladder cancer cells, brain cancer cells, or ovarian cancer cells. In some cases, the method can also be used to stage a cancer in a subject with respect to the extent of the disease and to monitor changes in response to therapy. In some embodiments, the cancer is breast cancer, liver cancer, lung cancer, prostate cancer, colon cancer, rectal cancer, stomach cancer, pancreatic cancer, esophageal cancer, kidney cancer, bladder cancer, brain cancer, or ovarian cancer.

A modified phage of the present disclosure can also be used to localize a tumor in vivo by administering to a subject a modified phage of the present disclosure, labeled with a reporter group giving off a detectable signal.

Cells that are bound to modified phages can then be detected using flow cytometry, microscopy, external scintigraphy, emission tomography, optical imaging, or radionuclear scanning. Detection of cells can be facilitated by coupling the aptamer or the modified phage to a detectable label or moiety. Examples of detectable labels include magnetic labels, various enzymes, prosthetic groups, fluorescent materials, luminescent materials, bioluminescent materials, chemiluminescent probes, metal particles, non-metal colloidal particles, electron dense labels, labels for MRI, radioactive materials, polymeric dye particles, pigment molecules, electrochemically actives species, semiconductor nanocrystal or other nanoparticles including quantum dots or gold particles. Examples of suitable enzymes include horseradish peroxidise, alkaline phosphatise, β-galactosidase, or acetylcholinesterase. Examples of suitable prosthetic group complexes include streptavidin/biotin and avidin/biotin. Examples of suitable fluorescent materials include fluorescein (FAM), umbellifone, fluorescein isothiocyanate, rhodamine, dischlorotriazinylamine fluorescein, dansyl chloride or phycoerythrin. Examples of fluorescent protein labels include green fluorescent protein (GFP) and variants thereof (e.g., cyan fluorescent protein and yellow fluorescent protein). Fluorescent labels include without limitation a rare earth chelate (e.g., europium chelate), rhodamine; fluorescein types including without limitation FITC, 5-carboxyfluorescein, 6-carboxy fluorescein; a rhodamine type including without limitation TAMRA; dansyl; Lissamine; cyanines; phycoerythrins; Texas Red; Cy3, Cy5, dapoxyl, NBD, Cascade Yellow, dansyl, PyMPO, pyrene, 7-diethylaminocoumarin-3-carboxylic acid and other coumarin derivatives, Marina Blue™, Pacific Blue™, Cascade Blue™, 2-anthracenesulfonyl, PyMPO, 3,4,9,10-perylene-tetracarboxylic acid, 2,7-difluoro fluorescein (Oregon Green™ 488-X), 5-carboxyfluorescein, Texas Red™-X, Alexa Fluor 430, 5-carboxytetramethylrhodamine (5-TAMRA), 6-carboxytetramethylrhodamine (6-TAMRA), BODIPY FL, bimane, and Alexa Fluor 350, 405, 488, 500, 514, 532, 546, 555, 568, 594, 610, 633, 647, 660, 680, 700, and 750, and derivatives thereof, among many others. See, e.g., “The Handbook—A Guide to Fluorescent Probes and Labeling Technologies,” Tenth Edition, available on the internet at probes (dot) invitrogen (dot) com/handbook. The fluorescent label can be one or more of FAM, dRHO, 5-FAM, 6FAM, dR6G, JOE, HEX, VIC, TET, dTAMRA, TAMRA, NED, dROX, PET, BHQ, Gold540 and LIZ. An example of a luminescent material includes luminol. Examples of bioluminescent materials include luciferase, luciferin, and aequorin, and examples of suitable radioactive material include ¹²³I, ¹²⁴I, ¹²⁵I, ¹³¹I, ³⁵S, ³²P, ¹⁸F, ⁶⁴Cu, ⁹⁴mTc, ⁹⁹Tc, ¹⁴C, ¹¹C, ¹³N, ¹⁵O, ⁶⁸Ga, ⁸⁶Y, ⁸²Rb, ¹¹¹In, ¹³³Xe, ¹⁷⁷Lu, ²¹¹At, ²¹³Bi, or ³H.

Using conventional techniques, the modified phage or the aptamer can be directly or indirectly labeled. Labeling at the 3′ end of the aptamer can be achieved, for example, by templated extension using Klenow polymerase, by T4 ligase-mediated ligation, and by terminal deoxynucleotidyl transferase. Labeling at the 5′ end can be achieved by the supplementation of the in vitro transcription mix with an excess of GTP-β-S, the thiol of which can then be used to attach biotin. In addition, direct chemical conjugation of a suitable group(s) to either 5′- or 3′-end can be used to label the aptamers. In some embodiments, the label is attached to the modified phage or the aptamer through biotin-streptavidin (e.g., synthesize a biotinylated aptamer, which is then capable of binding a streptavidin molecule that is itself conjugated to a detectable label; non-limiting example is streptavidin, phycoerythrin conjugated (SAPE)). Methods for chemical coupling using multiple step procedures include biotinylation, coupling of trinitrophenol (TNP) or digoxigenin using for example succinimide esters of these compounds. Biotinylation can be accomplished by, for example, the use of D-biotinyl-N-hydroxy succinimide. Succinimide groups react effectively with amino groups at pH values above 7, and preferentially between about pH 8.0 and about pH 8.5. Alternatively, the M13 phage or the aptamer is not labeled, but is later contacted with an antibody that comprises a detectable label is labeled after the aptamer is bound to target of interest.

Various enzyme-substrate labels may also be used in conjunction with a modified phage, e.g., modified M13 phage, or an aptamer disclosed herein. Such enzyme-substrate labels are available commercially (e.g., U.S. Pat. No. 4,275,149). The enzyme generally catalyzes a chemical alteration of a chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase (AP), β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of enzyme-substrate combinations include, but are not limited to, horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethylbenzidine hydrochloride (TMB)); alkaline phosphatase (AP) with para-nitrophenyl phosphate as chromogenic substrate; and β-D-galactosidase (β-D-Gal) with a chromogenic substrate (e.g., p-nitrophenyl-β-D-galactosidase) or fluorogenic substrate 4-methylumbelliferyl-P-D-galactosidase.

A modified phage, e.g., modified M13 phage, of the present disclosure comprising an aptamer as disclosed herein can be used in a method for detection, isolation, enrichment, and/or characterization of cells, e.g., CTCs, that includes immobilizing the modified phage on a solid surface. In some embodiments, the modified phage is contacted with cells of interest, e.g., cancer cells in a biological sample extracted from a patient, under conditions that allow the modified phage to bind to its target, such as a cancer-related antigen on the surface of a cell. In some embodiments, the modified phage can be immobilized on a solid surface, and any cell that is tethered to the modified phage can be isolated and/or enriched. In some embodiments, the isolated cells are released from the modified phage.

In some embodiments, the presence or absence of binding in a complex mixture, e.g., a biological sample from a subject, such as a blood sample, is detected. For example, a threshold binding signal can be established such that higher signal than the threshold indicates binding to the modified phage or the aptamer on a particular solid surface. Alternatively, binding specificity can be determined and selected by identifying an aptamer on a modified phage that binds to a target cancer-related antigen but does not significantly (or has reduced binding) to a non-target molecule (e.g., an isoform of the target cancer-related antigen or a molecule similar but not identical to the target cancer-related antigen). In some embodiments, the target cancer-related antigen and background proteins may be labeled with distinct fluorophores, such that the on- and off-target binding of every aptamer can be characterized individually.

A modified phage of the present disclosure can also be used to isolate and/or enrich a cell, e.g., a cancer cell or a tumor cell, by administering to a subject a modified phage of the present disclosure, or by incubating in vitro a biological sample with a modified phage of the present disclosure. In some embodiments, the modified phage, e.g., modified M13 phage, comprises an affinity tag for an ion-functionalized solid surface. In some embodiments, the affinity tag is a six-histidine peptide (6His tag) on a capsid protein of the modified phage, e.g., a pIII capsid protein, and the ion-functionalized surface is a magnetic surface, such as a magnetic bead. In some embodiments, a magnet is used to isolate and/or enrich the cells. Modified phage immobilization on a solid surface is discussed in detail above.

In some embodiments, the isolated and/or enriched cells are analyzed for cancer cell subtype. In general, a cancer subtype is defined based on certain characteristics of the cancer cells, for example, morphology and/or the presence or absence or particular biomarkers. In some embodiments, the analysis of cancer cell subtype informs the diagnosis and/or prognosis of a disease. In some embodiments, the analysis of cancer cell subtype informs the types of treatments that can be recommended to a patient.

In some embodiments, the method for isolation, enrichment, detection, and/or characterization of CTCs comprises a bead-based method that can be used with a microfluidic device. For example, the labeled modified phage or aptamer can be immobilized to a bead or other solid surface and the binding reaction between the modified phage or aptamer and its target on a cell, e.g., a cancer-related antigen, is performed in a microfluidic device. Different compartments can comprise different modified phages and/or aptamers for different targets, and/or different biological samples. In this way, multiplexing can be performed using a microfluidic device. After the binding reaction between the modified phage or aptamer and its target, the cells isolated from the biological sample can be delivered to a detection device. The detection device, such as a dual or multiple laser detection system can be part of the microfluidic system and can use a laser to identify each bead or microsphere by its color-coding, and another laser can detect the binding signal associated with each bead.

In some embodiments, the isolation, enrichment, detection, and/or characterization of CTCs may be achieved using a modified phage or the present disclosure in a flow cytometry-based process. In some embodiments, the modified phage is linked to a detectable label and the isolated and/or enriched cells can be visualized, sorted, and/or quantitated, and flow cytometry can be used for sorting the particles suspended in a stream of fluid. Labeled cells can be separated into populations from an original mix, such as a biological sample, with a high degree of accuracy and speed. Flow cytometry allows simultaneous multiparametric analysis of the physical and/or chemical characteristics of single cells flowing through an optical/electronic detection apparatus. A beam of light, usually laser light, of a single frequency (color) is directed onto a hydrodynamically focused stream of fluid. A number of detectors are aimed at the point where the stream passes through the light beam; one in line with the light beam (Forward Scatter or FSC) and several perpendicular to it (Side Scatter or SSC) and one or more fluorescent detectors.

Flow cytometers can analyze several thousand particles every second in “real time” and can actively separate out and isolate particles having specified properties. They offer high-throughput automated quantification, and separation, of the set parameters for a high number of single cells during each analysis session. Flow cytometers can have multiple lasers and fluorescence detectors, allowing multiple labels to be used to more precisely specify a target population by their phenotype. Thus, a flow cytometer, such as a multicolor flow cytometer, can be used to detect one or more CTCs with multiple fluorescent labels or colors. In some embodiments, the flow cytometer can also sort or isolate different CTC populations, such as by size or by different markers.

b. Methods of Treatment

The aptamer-decorated phage can be used to deliver drugs, imaging probes or radioactive moiety to tumor for cancer therapy or imaging through intravenous or local injection at a dose of 0.01 mg/kg to 10 mg/kg body weight daily. The drugs can be loaded on the surface of phage by chemical conjugation, physical adsorption, or through loading drug-encapsulated nanoparticles such as liposomes. The aptamer-decorated phage can also be used to carry imaging probes such as iron oxide nanoparticles or bismuth nanoparticles to enhance the contrast of MRI or CT imaging. The aptamer-decorated phage can also be used to carry radioactive moiety such as fluorine-18 to tumor to enhance the PET imaging.

### V. Compositions and Kits

The present disclosure further provides a pharmaceutical composition comprising a modified phage, e.g., modified M13 phage, of the present disclosure comprising an aptamer as disclosed herein. In some embodiments, the aptamer is linked to a detectable moiety or therapeutic agent as described herein. A pharmaceutical composition of the present disclosure includes a composition prepared for storage, use, or administration that includes a pharmaceutically effective amount of the modified phage in a pharmaceutically acceptable carrier and/or excipient. The choice of excipient or other elements of the composition can be adapted in accordance with the route and device used for administration.

Suitable carriers that may be used in a composition include those conventionally used, for example, water, saline, aqueous dextrose, lactose, Ringer's solution a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like. Other general additives such as anti-oxidative agent, buffer solution, bacteriostatic agent etc. can be added. In order to prepare injectable solutions, pills, capsules, granules, or tablets, diluents, dispersing agents, surfactants, binders and lubricants can be additionally added.

The modified phages of the disclosure and formulations thereof may be administered systemically (e.g., by oral ingestion or by injection intravenously, intramuscularly, or subcutaneously) or administered directly or topically (e.g., locally) to the patient or target tissue or organ as is generally known in the art. For example, a composition can comprise a delivery vehicle, including liposomes, for administration to a subject. Phages can be administered to cells by a variety of methods known to those of skill in the art, including, but not restricted to, encapsulation in liposomes, by iontophoresis, or by incorporation into other vehicles, such as biodegradable polymers, hydrogels, cyclodextrins poly (lactic-co-glycolic) acid (PLGA) and PLCA microspheres, biodegradable nanocapsules, and bioadhesive microspheres, or by proteinaceous vectors.

Delivery systems to be used with the modified phages of the present disclosure include, for example, aqueous and non-aqueous gels, creams, multiple emulsions, microemulsions, liposomes, ointments, aqueous and non-aqueous solutions, lotions, aerosols, hydrocarbon bases and powders, and can contain excipients such as solubilizers, permeation enhancers (e.g., fatty acids, fatty acid esters, fatty alcohols and amino acids), hydrophilic polymers (e.g., polycarbophil and polyvinylpyrolidone), and the like. In some cases, the pharmaceutically acceptable carrier is a liposome or a transdermal enhancer.

A pharmaceutical composition of the disclosure is in a form suitable for administration, e.g., systemic or local administration, into a cell or subject, including a mammal such as a human being. Suitable forms, in part, depend upon the use or the route of entry, for example, oral, transdermal, or by injection. Other factors are known in the art, and include considerations such as toxicity and forms that prevent the composition from exerting its effect.

The aptamer or composition comprising a modified phage of the present disclosure can be administered parenterally (for example, intravenous, hypodermic, local or peritoneal injection). The effective dosage of the modified phage can be determined according to weight, age, gender, health condition, diet, administration frequency, administration method, excretion and severity of a disease. Generally, an amount between 0.01 mg/kg and 100 mg/kg body weight/day of the aptamer is administered.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin, or olive oil.

Aqueous suspensions may contain a modified phage of the present disclosure in a mixture with excipients suitable for the manufacture of aqueous suspensions. Such excipients are suspending agents, for example, sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example, polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example, polyethylene sorbitan monooleate. The aqueous suspensions can also contain one or more preservatives, for example, ethyl, or n-propyl p-hydroxybenzoate, one or more coloring agents, one or more flavoring agents, and one or more sweetening agents, such as sucrose or saccharin.

Oily suspensions can be formulated by suspending a modified phage of the present disclosure in a vegetable oil, for example, arachis oil, olive oil, sesame oil or coconut oil, or in a mineral oil such as liquid paraffin. The oily suspensions can contain a thickening agent, for example, beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the aptamer in admixture with a dispersing or wetting agent, suspending agent and one or more preservatives. Suitable dispersing or wetting agents or suspending agents are exemplified by those already mentioned above. Additional excipients such as sweetening, flavoring and coloring agents can also be present.

Pharmaceutical compositions can also be in the form of oil-in-water emulsions. The oily phase can be a vegetable oil or a mineral oil or mixtures of these. Suitable emulsifying agents can be naturally-occurring gums, for example, gum acacia or gum tragacanth, naturally-occurring phosphatides, for example, soy bean, lecithin, and esters or partial esters derived from fatty acids and hexitol, anhydrides, for example, sorbitan monooleate, and condensation products of the partial esters with ethylene oxide, for example, polyoxyethylene sorbitan monooleate. The emulsions can also contain sweetening and flavoring agents.

A sterile injectable preparation can also be a sterile injectable solution or suspension in a non-toxic parenterally acceptable diluent or solvent, for example, as a solution in 1,3-butanediol. Among the acceptable vehicles and solvents that can be employed are water, Ringer's solution, isotonic sodium chloride solution, and an isotonic salt solution containing sodium and potassium chloride. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy. The administration frequency may be one to several times a day, weekly, or monthly.

Also provided herein are kits for isolating and/or detecting cancer cells. A kit can include a modified phage, e.g., modified M13 phage, of the present disclosure comprising an aptamer as disclosed herein, including as non-limiting examples, one or more reagents useful for preparing molecules for use in a method of the present disclosure, and instructions for use. In some embodiments, the modified phage comprising an aptamer is immobilized on a solid surface such as magnetic bead. In some embodiments, the kit further comprises a magnet for use with the magnetic beads.

### EXAMPLES

The following examples are provided by way of illustration only and not by way of limitation. Those of skill in the art will readily recognize a variety of non-critical parameters that could be changed or modified to yield essentially the same or similar results.

### Example 1—Materials and Methods

Cell lines. MCF-7 (Cat. #: TCHu 74), HepG2 (Cat. #: TCHu 72) and A549 (Cat. #: TCHu150) cell lines were supplied by Stem Cell Bank, Chinese Academy of Sciences (China). MCF-10A (Cat. #: CL-0212), MDA-MB-231(Cat. #: CL-0150B), Ramos (Cat. #: CL-0483) and SK-Hep-1 (Cat. #: CL-0525) cell lines were obtained from Procell (China). All these cells were cultivated in Dulbecco's Modified Eagle Medium (DMEM) (Gibco, USA) supplemented with 10% fetal bovine serum (FBS) (Gibco, USA) and 1% antibiotics (Gibco, USA) at 37° C. in 5% CO₂.

Propagation and purification of M13phage. M13 phage was propagated in Luria-Bertani (LB) medium using E. coli ER2738 (NEB, USA) as a host strain. The phages were first incubated with the bacteria culture at mid logarithmic growth phase for 5 h (MOI=0.1, 37° C., 250 rpm) and then separated from the host bacteria by centrifugation and concentrated by PEG/NaCl precipitation (20% (w/v) PEG-8000, 2.5 mol L⁻¹ NaCl) and suspended in Tris-HCl buffer (25 mmol L⁻¹ Tris-HCl, pH 7.5, 150 mmol L⁻¹ NaCl).

Genetic modification of M13 phage. For M13 phage tip modification (pIII capsid protein), 6His tag was displayed to immobilize M13 phages on Ni-functionalized solid surface in an end-on way, due to its high chelating efficiency with Ni atom, moderate stability of this chelation, and the ease in operation. See, e.g., Alarcó n-Correa, M. et al. ACS Nano. 13, 5810-5815 (2019); Shen, W. et al. J. Am. Chem. Soc. 131, 6660-6661 (2009); and Conti, M. et al. Angew. Chem. Int. Ed. 112, 221-224 (2000). In order to display 6His tag on the pIII protein, modified M13KE vector encoding 6His tag was constructed, as previously discussed in Cao, Y. et al. Small 18, 2203962 (2022). To be specific, two synthetic phosphorylated oligonucleotide strands, 6H-Tag1/6H-Tag2 (see Informal Sequence Listing for sequences), were hybridized by annealing at 95° C. for 5 min. M13KE vector (NEB, USA) was linearized by double-digestion with Acc65I and EagI(NEB, USA), followed by purification using a gel purification kit (NEB, USA). The purified product was ligated with the hybridization product at 16° C. for 12 h. The ligation product was then transferred into chemical competent E. coli TG1 cells (Lucigen, USA) by CaCl₂) transformation method. The transformed E. coli was grown overnight on LB/IPTG/X-Gal agar plates. Colonies were picked and validated by DNA sequencing.

Hardening treatment of 6His-M13 phage. For evaluating the impact of flexibility of M13 phages on cell isolation performance, 6His-M13 phage was incubated with equal volume of 4% paraformaldehyde (PFA) solution (6 h, 4° C., Dalian Meilun Biotechnology, China) and ultrafiltered for subsequent reactions, resulting in the formation of PFA-treated M13. Alternatively, 6His-M13 phage was also hardened by treatment with ethanol (100%, EtOH), generating EtOH-treated M13. The hardening treatment by EtOH was the same with that by PFA. Both PFA-treated M13 and EtOH-treated M13 were further subjected to the same procedure of azide modification and aptamer decoration. The amount of loaded aptamer on the PFA-treated rigid M13 was measured to be almost identical to that of the untreated flexible M13 (see Table 1 in Example 4 below).

Azide modification of 6His-M13phage. The 6His-M13 phages were chemically modified to incorporate azide groups by coupling with an active ester to form an amido bond in sterile PBS (pH 7.8) according to a modified protocol previously discussed in Yuan, R. et al. Anal. Chem. 91, 4948-4952 (2019); and Jeon, C. S. et al. Adv. Funct. Mater. 23, 1484-1489 (2013). Specifically, the phages (10¹¹ pfu) were dissolved in 50 mM PBS (240 L) with gentle mixing, followed by the addition of 250 μL N₃-PEG-NHS (10 mM, dissolved in DMSO, MeloPEG, China). After 18 hours at 4° C. and shaking at 200 rpm, excess N₃-PEG-NHS was removed by ultrafiltration (molecular weight cutoff (MWCO)=100 kDa, Millipore, USA) and three washes with phosphate buffer saline (PBS). The resulting N₃-M13 was collected and stored at 4° C. for future use. Both N3-M13 and unmodified M13 were desalted by ultrafiltration (MWCO=100 kDa, Millipore, USA) and injected into Q-TOF LC/MS (Agilent Technologies, 1260-6540) for modification characterization.

Aptamer-loading on N₃-M13 phage-immobilized magnetic microbeads (MBs). In general, 1 mL of Ni-iminodiacetic acid (Ni-IDA) functionalized magnetic MBs (2.5 mg mL-1, Solarbio, China) were repeatedly washed with PBST (1×PBS, 3% bovine serum albumin (BSA), tween-20 (0.01% (v/v), pH 7.4) followed by a 3-h incubation with 5×10¹⁰ pfu of N3-M13 with gentle rotating at room temperature (Alarcó n-Correa, M. et al. ACS Nano. 13, 5810-5815 (2019)). Subsequently, the N₃-M13 phage-anchored MBs (N3-M13-MBs) were repeatedly rinsed with PBST and resuspended in 100 μL PBS for further aptamer decoration.

Dibenzocyclooctyne (DBCO)-functionalized aptamer S2.2 (DBCO-Apt; SEQ ID NO: 1; see Informal Sequence Listing below; Ferreira, C. S. M. et al. Tumor Biol 27, 289-301 (2006)) was used for specific targeting of MUC1 positive cell lines. Aptamer decoration was achieved via click chemistry between DBCO-Apt (SEQ ID NO: 1) and N₃-M13. In brief, 100 μL of N₃-M13-MBs were allowed to react with 100 μL of DBCO-Apt (2.5 M) for 6 h (200 rpm, 25° C.). The product of aptamer-decorated flexible M13-MBs (A-f-M13-MB) was repeatedly rinsed and resuspended in 500 μL PBS buffer. The counterpart MBs produced with rigid M13 phages was denoted as for the comparison of cell isolation performance.

Conjugation of aptamers on magnetic microbeads (MBs). Aptamers were conjugated on magnetic MBs by a single-step carbodiimide reaction between NH₂-terminated aptamer and carboxyl MBs (Solarbio, China) (Wu, N. et al. Anal. Chem. 92, 11111-11118 (2020)). Briefly, 500 μL of carboxyl MBs (2.5 mg mL⁻¹, suspended in 100 mM MES buffer, Sigma-Aldrich, USA) was activated by the addition of 250 μL EDC (50 mg mL⁻¹, Aladdin, China) and an equal volume of NHS (20 mg mL⁻¹, Sangon Biotechnology, China). The mixture was incubated for 60 min at 25° C. on a rotator with shaking at 250 rpm. The activated MBs were magnetically separated and repeatedly washed with PBS. Afterward, 24 μL of aptamer (10 M) were incubated with MBs suspension overnight (25° C.). The product of aptamer-decorated MBs (A-MB) was obtained by magnetic separation and stored at 4° C. The amount of aptamers decorated on MBs was estimated by decorating MBs with FAM-labeled aptamer and comparing the difference between the fluorescence of input FAM-aptamer-NH₂ and the fluorescence of the supernatant after the decoration reaction.

Aptamer-loading on N₃-M13. A total of 10¹⁰ pfu of N₃-M13 was reacted with 1.25 M (final concentration) of DBCO-aptamer (SEQ ID NO: 1) in PBS for 6 h (25° C.). Afterwards, the mixture was purified by PEG/NaCl precipitation for six hours at 4° C. Purified Apt-M13 was obtained with a 10-min centrifugation step (9391×g).

Gel electrophoresis analysis and western blotting (WB) analysis. Polyacrylamide gel electrophoresis (PAGE) was conducted to characterize the assembly of Y-shaped DNA scaffold. A total of 10 μL DNA strand (1 μM) was added into 2.5 μL of 1× loading buffer at 95° C. for 5 min. The electrophoresis was carried out using 12% polyacrylamide gel and 1×Tris-Boric acid-EDTA running buffer (100 V, 60 min, 4° C.). The gels were further stained using 4S Red Plus Nucleic Acid Stain (Sangon Biotechnology, China) and characterized by Tanon-4600SF automatic digital gel imaging analysis system (China).

For the evaluation of protein expression in the cells before and after isolation and re-culture, CTC cells (MCF-7 as model cell, cell number: 5×10⁶, Stem Cell Bank, Chinese Academy of Sciences) were lysed by RIPA buffer (Epizyme, China) and protein was extracted. Afterwards, 20 μg of cellular protein (quantified by BCA protein detection kit, Epizyme, China) was loaded on an 8˜12% gel for separation by SDS-PAGE. The proteins were transferred to a 0.22 μm-polyvinylidene fluoride (PVDF) membrane (Cat. #: WJ001S, Epizyme, China) and blocked with 5% skim milk at room temperature for 60 min to prevent non-specific binding. Then, the anti-ER antibody (Cat. #: ab32063, clone: E115, 1:500 dilution, Abcam, USA) and anti-GAPDH antibody (Cat. #: ab9485, clone: pAb, 1:2500 dilution, Abcam, USA) were diluted with 5% skim milk and separately added for overnight-incubation at 4° C. The membrane was then washed six times with TBST (0.5% (v/v) tween 20) and incubated with the secondary antibody (HRP Conjugated AffiniPure Goat Anti-rabbit IgG (H+L) antibody, Cat. #: BA10541, clone: NA, 1:8000 dilution, Boster, China) at room temperature for 60 min. The membrane was washed with TBST and incubated with enhanced chemiluminescence (ECL) reagents (Epizyme, China) for chemiluminescence imaging (Bio Rad, USA). Quantitative analysis was conducted using ImageJ software.

Young's modulus and stiffness assay of M13 phage. Briefly, about 10 μL M13 phage (1010 pfu mL-1) was pipetted onto mica discs and left to stand for about 5 min. Excessive solvent was removed with a filter paper. Thereafter, deionized (DI) water was pipetted three times to remove excessive salt and the remaining water was evaporated slowly at room temperature. Young's modulus and stiffness were measured using the contact mode using Bruker Dimension icon atomic force microscopy (AFM, Bruker, Dimension icon). The actual bending of the cantilever representing deflection sensitivity and spring contact of the cantilever measured in the air was calibrated. Then, 5 points along a M13 phage nanofiber were selected to measure the Young's modulus and stiffness the phage. At least ten phage samples were measured, and the recorded measurements were averaged.

Numerical simulations. Numerical simulations were conducted using COMSOL (version 5.6, COMSOL Ltd, USA) to simulate the force induced deformation of M13 nanofibers with different stiffnesses. The computing domain was set as 3×3 μm. An M13 nanofiber was 880 nm in length (Y direction), 6 nm in width (X direction), and the elastic modulus E was 0.5 GPa and 1.35 GPa for flexible and rigid M13, respectively. An M13 nanofiber was modeled as a linear elastic solid material. The direction of the flow field was set from the left boundary to the right with the inlet normal velocity given by Eq. (1). No-slip boundary conditions were imposed at all the other boundaries. Automatic remeshing was used to capture the dynamic motion of the M13 nanofibers.

$${u}_{i n} = \frac{U \times {t}^{2}}{\sqrt{{( 0.04 - {t}^{2} )}^{2} + {( 0.1 t )}^{2}}} \qquad ( 1 )$$

Determination of the binding affinity toward cancer cells. To assess the binding affinity of A-f-M13-MB toward MUC1 positive cells, 5×104 MCF-7 cells were first pre-stained with 4′,6-Diamidino-2′-phenylindole (DAPI, Boster, China) and then added into 500 μL of A-f-M13-MB suspension for 30 min-incubation at 37° C. After incubation, the fluorescence intensity of the unbound cells in the supernatant was measured by a microplate reader (Synergy H1, BioTek, USA) at λ_{Ex}/λ_{Em}=360 nm/460 nm. Fluorescence from the captured cells was estimated by subtraction. By plotting the fluorescence intensity (FL) with the concentration of Apt (C_{Apt}), the binding affinity curve was obtained, which fitted the equation FL=Bₘₐₓ C_{Apt}/(K_{d}+C_{Apt}), where Bₘₐₓ refers to the maximum amount of binding sites, and K_{d} refers to the binding constant, with a lower K_{d} indicating a higher binding affinity. The K_{d} of MCF-7 by A-r-M13-MB and A-MB was also measured using the same method to compare their CTC binding affinity. As for free aptamer, instead of collecting the fluorescence intensity of CTCs, fluorescence from FAM-aptamer (SEQ ID NO: 4) was used and thus the K_{d} value could be calculated similarly (Wang, S. et al. Anal. Chem. 94, 9450-9458 (2022)).

Preparation of Nickel-Iminodiacetic Acid (Ni-IDA) grafted glass slides for M13 phage grafting. The Ni IDA-grafted glass slides were prepared as discussed in Liu, J. J. Mater. Chem. B, 1, 810-818 (2013). First, the glass slides (1×1 cm²) were first treated with piranha solution (an etching solution comprised of sulfuric acid and hydrogen peroxide) to activate hydroxyl groups, and then placed in 5 mL tubes and immersed in alkaline solution for 20 min. They were then immersed into 170 μL epichlorohydrin (Tianjin Damao Chemical Reagent, China) under ultrasonic bath for 30 min for epoxy-functionalization. Next, the epoxy glass slides were reacted with iminodiacetic acid (Sinopharm, China) at 70° C. for 10 h and further chelated with nickel ion (0.1 M) at 25° C. for 3 h. The glass slides were carefully rinsed with DI water between every two steps. The resulting Ni-IDA grafted glass slides were capable of anchoring 6His-M13 or N₃-M13 via 6His-tagged pIII protein in an end-on manner. After grafting of N3-M13 (1010 pfu) on Ni-IDA grafted glass slide, DBCO-Apt (SEQ ID NO: 1) was clicked on the slide to produce an “A-f-M13-slide.” The procedure used was as described above under the subheading “Aptamer-loading on N₃-M13 phage-immobilized magnetic microbeads (MBs).” The counterpart slide grafted with rigid M13 phages was denoted as “A-r-M13-slide” and used for the comparison of cell adhesion force. For the preparation of the aptamer-decorated slide, “A-slide,” FAM-aptamer-NH₂ (SEQ ID NO: 4) was used to conjugate with the epoxy glass slides after a 10 h-reaction at 25° C.

Cell adhesion force measurements. The adhesion force between cells and M13 nanofibers was evaluated by a reversed centrifugation force method as discussed previously in Jiang, W. et al. Adv. Sci. 7, 2002259 (2020). Briefly, 104 MCF-7 cells (or 106 Ramos cells, Procell, China) were pre-stained with acridine orange (AO; Beyotime, China) and incubated with A-f-M13-slide in a 24-well plate for 30 min. Uncaptured cells were gently washed off from the slide with PBS buffer, and remaining cells on the slide was counted under the fluorescence microscope (at 10× objective lens magnification). Next, the slide was inverted and placed on the bottom of a 15 mL centrifuge tube that was filled with 10 mL PBS and centrifuged with centrifugal forces ranging from 33 to 1905×g for 5 min. After that, the number of the remaining cells on the slides was enumerated under fluorescence microscope. The percentage of remaining cells on the slide at different centrifugal force was calculated as follows:

$$Remaining cells \% = \frac{Number of remained cells after centrifugation}{Number of cells before centrifugation} \times 100 \% \qquad ( 2 )$$

The cell adhesion force was equal to the relative centrifugal force when 50% cells remained on the slide. The relative centrifugal force of cells can be calculated using the following equation:

$${F}_{c} = ( {ρ}_{cell} - {ρ}_{m e d i u m} ) \times {V}_{cell} \times {ω}^{2} \times ( {r}_{0} + x ) \qquad ( 3 )$$

where F_{c} is the relative centrifugal force, ρ is the density of cells or 1×PBS medium (the cell density is about 1.07 g cm⁻³ and the PBS medium density is about 1 g cm⁻³), V_{cell} is the volume of cell (4000 μm³ for MCF-7 cells and 500 μm³ for Ramos cells), ω and r₀ are the angular speed and radius of centrifugation, x is the lateral distance from the bottom of the tube to the center of centrifuge. In order to get a fair comparison, the amount of loaded aptamers was kept the same for A-f-M13-slide, A-r-M13-slide, and A-slide.

Molecular modeling. Dissipative particle dynamics (DPD) simulations were used to better understand the interactions in the fluid between the receptors on CTCs and the aptamers on MB and A-f/r-M13-MB systems at the molecular level. Due to its large size, the MB was modeled as a planar substrate in the simulations. As a result, the A-MB was modeled as a planar substrate with the aptamer covalently decorating the substrate; the aptamer was modeled as a linear polymer made of beads with one bead at the end that can interact with the specific beads constituting the receptor. The total number of the aptamers in A-MB was 196. The A-f-M13-MB was modeled as a planar substrate with sixteen cylinders (with each cylinder representing a M13 phage) coated on its surface. The neighboring beads in the cylinder were connected by a harmonic bond to ensure its integrality, allowing the cylinder to freely twist or bend in the simulations. Twelve aptamers were covalently decorated on each cylinder, reaching 192 aptamers in A-f-M13-MB, similar to A-MB. The A-r-M13-MB was the same as the A-f-M13-MB except that the cylinder was treated as a rigid body, thus the cylinder was unable to freely twist or bend in the simulations. A white blood cell (WBC) was modeled as a lipid vesicle, which was fabricated by self-assembling of about 2000 lipid molecules. A CTC was modeled as a lipid vesicle with some receptors on its surface, where the lipid was the same as that in the WBC and the receptor was modeled as the branched polymer with one specific bead at the terminal of each branch.

DPD is a coarse-grained (CG) simulation technique with hydrodynamic interaction (Groot, R. D. & Warren, P. B. J. Chem. Phys. 107, 4423-4435 (1997)). The dynamics of the beads are governed by Newton's equation of motion. The total force exerted on each bead typically includes three parts:

$${F}_{j} = {\sum }_{i \neq j} ( {F}_{ij}^{C} + {F}_{ij}^{D} + {F}_{ij}^{R} ) where {F}_{ij}^{C}$$

is the conservative force;

$${F}_{ij}^{D}$$

is the dissipative force, and

$${F}_{ij}^{R}$$

is the random force. To denote the hydrophilic/hydrophobic property of the beads, the repulsive parameter aᵢⱼ was set in conservative force as 25 k_{B}T/r_{c} if i=j, and aᵢⱼ=100 k_{B}T/r_{c} if i≠j^{57,58}. Moreover, the “soft” Lennard-Jones (L) potential (Yang, K. & Ma, Y. Q. Nat. Nanotechnol. 5, 579-583 (2010)) was used to model the strong receptor-aptamer specific interaction and the weak lipid head-aptamer non-specific interaction. The interaction strength was set as 6.0 k_{B}T in the former case and it was set as 1.5 k_{B}T in the latter case. In addition, the harmonic spring interaction Uₛ=kₛ(r_{i,i+1}−l₀)² between neighboring beads in a single molecule was used to ensure the integrality of lipids, aptamers and M13^{57,58}, where kₛ is the spring constant and l₀ is the equilibrium length and the parameter kₛ=64 k_{B}T, l₀=0.5 r_{c} was used. Further, a three-body angle potential Uₐ=kₐ(1−cos(ϕ−ϕ₀)) was used to depict the rigidity of lipid tails and aptamer (kₐ=10.0 k_{B}T and ϕ₀=180), where kₐ is the energy constant and ϕ₀ is the equilibrium value of the angle. See, e.g., Alexeev, A., Uspal, W. E. & Balazs, A. C. ACS Nano. 2, 1117-1122 (2008), and Ding, H. M. et al. ACS Nano. 6, 1230-1238 (2012).

The velocity-Verlet integration algorithm was used to integrate Newton's equations of motion with the integration time step of 0.02,c. For simplicity, the bead mass m, the cutoff radius r_{c}, and the energy k_{B}T were chosen as the reduced units in the simulations. The simulations were performed in the NVT ensembles with the periodic boundary conditions adopted in the three directions. The size of the box was set as 60×60×50 and the number density of the beads was set as 3.0 in the simulations. All the simulations were carried out using the modified software package Lammps (12 Dec. 2018) (Plimpton, S. J Comput. Phys. 117, 1-19 (1995)).

Cell capture. The A-f-M13-MB was pre-blocked with a blocking solution (3% BSA in PBS) to reduce non-specific adsorption caused by bare MB surfaces before cell capture experiments. First, both the positive or negative cells were harvested using 0.25% trypsin and immediately resuspended in PBS buffer. Next, after enumeration with a hemocytometer, cell suspensions were diluted to the desired concentrations before use. Thereafter, the model cells were either resuspended in the PBS buffer, or stained with AO and resuspended in the whole blood samples. Afterwards, the cell suspension (500 μL) was incubated with equal volume of A-f-M13-MB for 30 min at 37° C. Subsequently, the uncaptured cells was magnetically separated and counted under fluorescence microscope (Olympus, BX53M) (Jo, S. M. et al. Small 11, 1975-1982 (2015)).

In order to evaluate the capture performance of A-f-M13-MB in a real sample matrix, a fixed number of positive cells were added to the freshly collected whole blood obtained from healthy volunteers, and the spiked sample was used to study the cell capture by A-f-M13-MB. The cell capture procedure was the same as described above, with the cell capture efficiency calculated using equation (4). All experiments were performed three times.

$$Cell captured \% = \frac{\begin{matrix}Toal number of the added cells - \\ Number of uncaptured cells\end{matrix}}{Number of input cells} \times 100 \% \qquad ( 4 )$$

Release assay, cell viability analyses and cell migration activity analyses. For the cell release assay, DNase I (Takara, China) was used to destroy the phosphodiester bond within the aptamers to release the captured cells. In order to determine the optimal release conditions, the dependance of the cell release efficiency on the concentration and incubation time of DNase I were investigated. Under optimized conditions, the captured cells from the buffer or the blood by A-f-M13-MB were magnetically separated, suspended in PBS buffer, and incubated for 20 min with 100 U mL⁻¹ DNase I at 37° C. Then, the cells in the supernatant were collected and enumerated. Cell release efficiency was derived using the following equation:

$$Cell released \% = \frac{Number of released cells}{Number of captured cells} \times 100 \% \qquad ( 5 )$$

Cell viability was evaluated by AO/propidium iodide (PI) assay (ApexBio, USA) according to manufacturer's instructions. The cells with high viability were stained by AO and emitted a green fluorescence, whereas dead cells were labeled by PI and emitted red fluorescence. Cell viability was calculated by determining counting the red and green emitting cells. To further validate the viability of released cells, cells were cultured at 37° C. and 5% CO₂ in DMEM cell culture medium.

For evaluating the migration activity of CTCs before and after isolation and re-culture, 6 mL of cell suspension (5×10⁴ cells mL⁻¹) was incubated in a 25 cm² culture flask for 3 h for cell adhesion. The Live cell Station (CytoSMART Lux3, Axion BioSystems, USA) were used to collect cell migration images at a fixed area every 5 min for 4 h. Subsequently, ImageJ (version 1.53t) was used for the analysis of the average moving distance and speed and the movement trajectory of cells was obtained using Matlab software (version R2020b).

CTCpurity during capture and release processes. Ramos cells were used as a white blood cell (WBC) model to investigate CTC purity during capture and release. WBCs were stained with DAPI and MCF-7 cells were stained with AO for discrimination. The cells were counted using a hemocytometer. Approximately 100-1000 MCF-7 cells were mixed evenly with 106 WBCs. Afterwards, A-f-M13-MB was mixed with the cell mixture for 30 min-incubation at 37° C. to capture target CTCs. Then, the uncaptured cells were collected and counted under fluorescence microscope. The captured cells were subsequently released using DNase I and counted. The number of nonspecifically attached WBCs were obtained from the number of initial input WBCs subtracting the number of uncaptured WBCs. The purity of CTC during capture and release processes was evaluated by depletion index value. The WBCs depletion index was calculated according to the following equation:

$$WBCs depletion = {log}_{10} ( \frac{Number of initial WBCs}{Number of final WBCs} ) \qquad ( 6 )$$

Study design. For evaluating the diagnostic efficacy of A-f-M13-MB, a total of 100 participants were enrolled in this study, including 56 breast cancer patients, 34 benign patients, and 10 healthy volunteers. As breast cancer is mostly suffered by female patients, most of the participants involved in this study were female (95%). Considering the possibility of male patients that suffer from benign breast disease, the cohort involved four healthy male donors and one male benign patient. All participants were recruited from Liaoning Cancer Hospital. Only patients with definite information of sex, age, and pathological diagnosis were recruited. The study complied with all relevant ethical regulations and was approved by the Ethics Committee of both Northeastern University, China (No. NEU-EC-2021B020S) and Liaoning Cancer Hospital (20211035). All individuals were anonymized, and only gender, age, pathological diagnosis, treatment plan and treatment response were recorded. The requirement for consent was waived by the ethical review body, as the samples collected were remnant samples destined for disposal on the day of sample request from the study. The consent to publish the participants' information regarding their age and sex were obtained. No self-selection criteria bias for patient populations was anticipated.

Isolation and analysis of CTCs from breast cancer patients. 500 μL of whole blood from each breast cancer patient (without any pre-treatment) was incubated with 500 μL A-f-M13-MB at 37° C. for 30 min with gentle shaking. After magnetic separation, the captured CTCs were released using 100 U mL-1 DNase I at 37° C. for 20 min. Finally, the released cells were identified using CTC immunofluorescence staining kit (IFH-001, Cytelligen, USA). Briefly, the released cells were fixed with 4.0% formaldehyde at 33° C. overnight. They were then blocked with 3% goat serum followed by incubation with antibody cocktail (EpCAM antibody conjugated with AF488, anti-CK conjugated with Cy5 and anti-CD45 conjugated with AF594) and DAPI for observation using a fluorescent microscope. The isolated CTCs in breast cancer patients' blood were identified as DAPI+/EpCAM+/CK+/CD45− cells, whereas WBCs were identified as DAPI+/EpCAM−/CK−/CD45+ cells. Meanwhile, to access the molecular profile of the captured cells, the anti-HER2 antibody (Cat. #: ab237060, clone: EP2324Y, 1:100 dilution, Abcam, USA), anti-estrogen receptor alpha antibody (Cat. #: ab205851, Clone: EPR4097, 1:50 dilution, Abcam, USA), anti-CD45 antibody, and DAPI were used according the same staining process. The luminal model cells were identified as DAPI+/ER+/HER2− or +/CD45− cells, HER2 model cells were identified as DAPI+/ER−/HER2+/CD45− cells, and triple-negative model cells were identified as DAPI+/ER−/HER2−/CD45− cells.

Isolation of CTC mixtures with different EMT subphenotypes by A-f-M13-MB bearing Y-shapedDNA scaffold. The Y-shaped DNA scaffold was assembled by annealing Y1, Y2 and Y3 (1.25 μM for each strand) at 95° C. for 6 min, and attached to 2.5 mg mL-1 1 mL of M13-MBs via click reaction following the same procedure described above under the subheading “Aptamer-loading on N3-M13 phage-immobilized magnetic microbeads (MBs).” The sequences of Y₁, Y₂ and Y₃ (SEQ ID NOS: 7-9) are disclosed in the Informal Sequence Listing below. For evaluating the CTC isolation performance of the resultant A-f-M13-MB, CTC mixtures (which contained 1,000 CTCs in total) were prepared by spiking MCF-7 cell (E-type CTC model) and MDA-MB-231 cell (M-type CTC model) in CTC-free whole blood at a molar ratio of 9:1, 3:1, 1:1, 1:3 and 1:9. The CTC mixtures were then captured following procedures described above under the subheading “Isolation and analysis of CTCs from breast cancer patients.” Additionally, immunofluorescence staining was applied using Anti-E Cadherin antibody (Cat. #: ab40772, clone: EP700Y, 1:1000 dilution, Abcam, USA) and Goat Anti-Rabbit IgG H&L (Alexa Fluor® 488) anti-body (Cat. #: ab150077, clone: NA, 1:200 dilution, Abcam, USA), Anti-N Cadherin antibody (Cat. #: ab98952, clone: 5D5, 1:200 dilution, Abcam, USA), and Goat Anti-Mouse IgG H&L (Alexa Fluor® 594) (Cat. #: ab150116, clone: NA, 1:200 dilution, Abcam).

Statistics and reproducibility. IBM SPSS Statistics software (version 19.0) was used to test the difference for diagnosis stages from different CTCs using a two-tailed t-test. The differences were considered significant at p<0.05 (**p<0.01, ***p<0.001, ****p<0.0001), and considered to have no significance at p>0.05 (ns). Confusion matrix for subtype diagnosis by CTC molecular profiling method was drawn by Origin software (version 2021). Receiver operating characteristic (ROC) curve was plotted using the MedCalc statistical software (version 20. 010), presenting the diagnostic accuracy. The results of dissipative particle dynamics simulations were analyzed by GraphPad prism (version 9.3.0). The mean distance and migration speed of cells before and after isolation and re-culture were collected using Image J software (version 1.53t) with Manual Tracking plugin. The movement trajectory of cells before and after isolation and re-culture was analyzed using Matlab software (version R2020b). All experiments were repeated independently with similar results for at least three times, especially micrograph results.

### Example 2—Phage Engineering and its Anchoring on Ni-IDA-Grafted Solid Surface

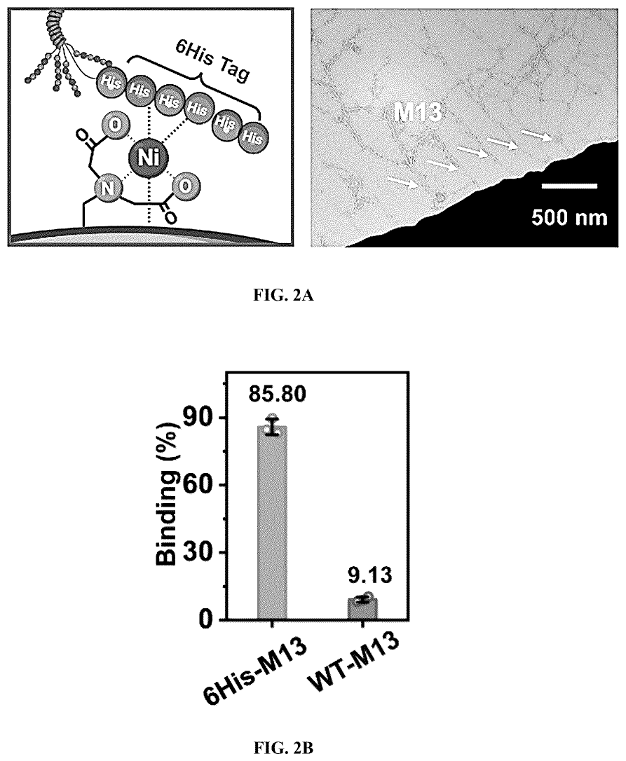

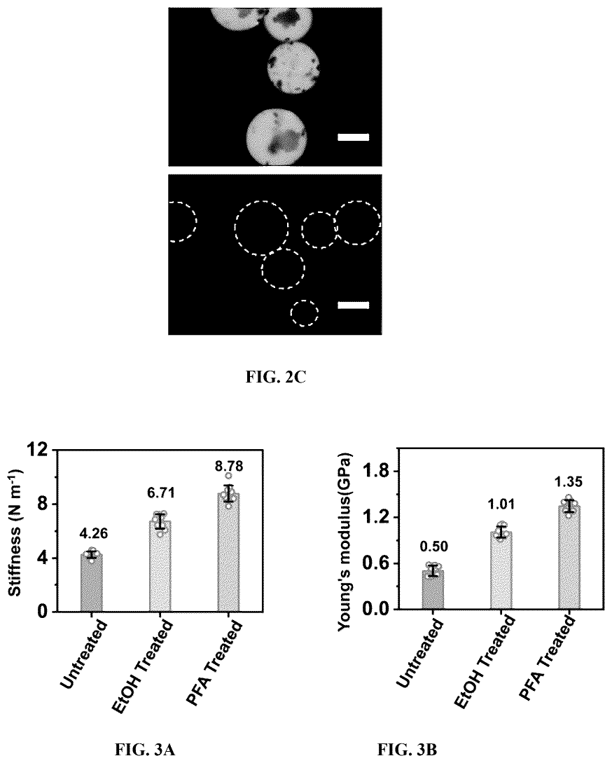

The M13 phage was subjected to genetic engineering to introduce a 6His tag at the N-terminus of the pIII minor capsid protein. This process, depicted in FIG. 1A, Step (i), resulted in the creation of a modified phage nanofiber known as 6His-M13. The strategic placement of the 6His tag at the phage's tip played a pivotal role in enabling precise immobilization of the M13 phage onto surfaces such as glass slides or magnetic microbeads (MBs). These surfaces had been modified with Ni-IDA and allowed for end-on immobilization (FIGS. 2A-2B).

To confer M13 with the ability to target CTCs, a CTC-specific aptamer known as S2.2 (see SEQ ID NO: 1), designed against the variable number tandem repeat (VNTR) region of Mucin 1 (MUC1) (Ferreira, C. S. M. et al. Tumor Biol. 27, 289-301 (2006)), was covalently linked to the exterior of the 6His-M13 phage. This linkage was achieved by introducing an azide functional group onto the phage's sidewall through a reaction involving NHS-PEG-N3 and the N-terminal amine (NH2) of the pVIII protein, as confirmed by Q-TOF LC/MS. This process, outlined in FIG. 1A, Step (ii), resulted in the creation of an azide-modified 6His-M13 phage termed N3-M13, with an impressive grafting ratio of approximately 85.80%. Subsequently, the N3-M13 phage was affixed to Ni-IDA MBs (FIG. 1A, Step (iii)), and then the dibenzocyclooctyne (DBCO)-labeled aptamer (DBCO-Apt; SEQ ID NO: 1) was efficiently linked to the phage's sidewall using a click reaction (FIG. 1A, Step (iv), FIG. 2C). This process yielded an assembly designated as an aptamer-flexible-M13-MB (A-f-M13-MB), with each individual phage carrying the modified aptamer (Apt-M13). An average of approximately 622 aptamers was found on the sidewall of each Apt-M13. This distribution represented roughly a quarter of the pVIII proteins functionalized with aptamers, and there was an average distance of approximately 10-12 nm between neighboring aptamers (Chen, L. et al. Chem. Commun. 51, 10190-10193 (2015)). Notably, considering the aptamer's size of about 2-3 nm, the density of aptamers on the M13 phage surface was well-arranged to prevent tangling or overlap between individual aptamers. The stability of A-f-M13-MB was also evaluated—A-f-M13-MB could be preserved and was stable in when stored its lyophilized form for over ˜7 weeks at −20° C., which underscores its potential use as a regular diagnostic tool in the clinic.

### Example 3—Mechanical Properties and Dynamic Movement of the Engineered M13 Phage

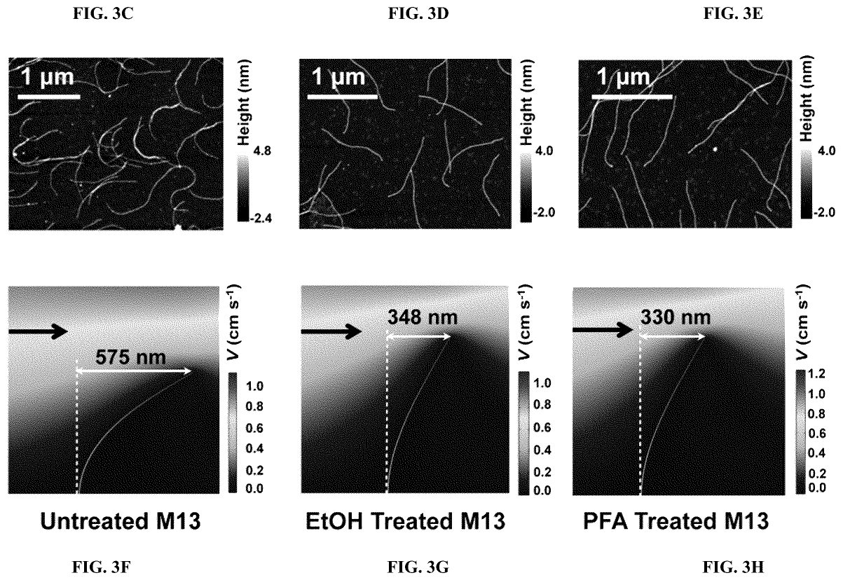

Atomic force microscopy (AFM) analysis confirmed that the Apt-M13 phage displayed a notably low Young's modulus (E) and exhibited soft characteristics, consistent with the proteinaceous composition inherent in M13 phages, which are recognized as pliant materials with much configurational freedom in flowing solutions (FIGS. 1C and 3A-3B). The tailored design of these phages, boasting high aspect ratios combined with their soft nature, facilitated pronounced twisting behavior within a solution (FIGS. 1C and 3C), underscoring their inherent flexibility.

To comprehensively investigate the implications of M13 phage flexibility and dynamic transformation on ensuing CTC capture efficiency, the rigidity of the M13 phage was enhanced, thereby restraining M13 mobility in solution. This involved treating the M13 phage with 4% paraformaldehyde (PFA) or 100% ethanol (EtOH). As a result, the treated, flexible M13 phages transformed into rigid M13 nanofibers, as evidenced by a substantial increase in their Young's modulus (2-3 times) and stiffness (˜1.5-2 times) (FIGS. 1C and 3A-3B). Notably, these rigid M13 nanofibers exhibited a less convoluted morphology than their untreated, flexible counterparts (FIGS. 3C-3E), and could only offer limited sites for binding CTCs. Meanwhile, the flexibility of untreated M13 nanofibers allowed for an increase in the binding free energy for the non-specific adsorption of WBCs due to entropy loss, thus guaranteeing a low WBC background (FIG. 1C). AFM images revealed that these treated rigid M13 presented a less twisty morphology than untreated M13 (FIGS. 3D-3E).

For better visualization of the flexibility difference between these phages in a moving state, a numerical simulation analysis was conducted to simulate the strain-induced deformation of M13 nanofibers with varying degrees of stiffness. The outcomes demonstrated that, under the same fluid shear stress, the deformation experienced by the untreated, flexible M13 nanofibers was 1.65-1.74 times greater than that exhibited by the treated, rigid counterparts (FIGS. 3F-3H). This disparity signified a greater spatial configurational freedom within the flexible M13 nanofibers relative to their rigid counterparts.

Given that PFA treatment rendered the M13 phages more rigid and did not impede subsequent aptamer decoration (i.e., sufficient NH₂— remained for aptamer decoration after PFA treatment) for simplicity, the PFA-treated M13 phage is referred to as “rigid M13” or “rigid Apt-M13,” and the untreated counterpart is referred to as “flexible M13” or “flexible Apt-M13.”

### Example 4—Flexible M13 Facilitates Better Capture of Circulating Tumor Cells (CTCs)

The augmented affinity observed in multivalent receptor-ligand interactions is primarily attributed to chelating and statistical rebinding effects. The potency of these effects largely hinges on the density and geometric arrangement of both receptors and ligands. See, e.g., Fasting, C. et al. Angew. Chem. Int. Ed. 51, 10472-10498 (2012); Bhatia, S. et al. J. Am. Chem. Soc. 138, 8654-8666 (2016); and Bachem, G. et al. Angew. Chem. Int. Ed. 59, 21016-21022 (2020).

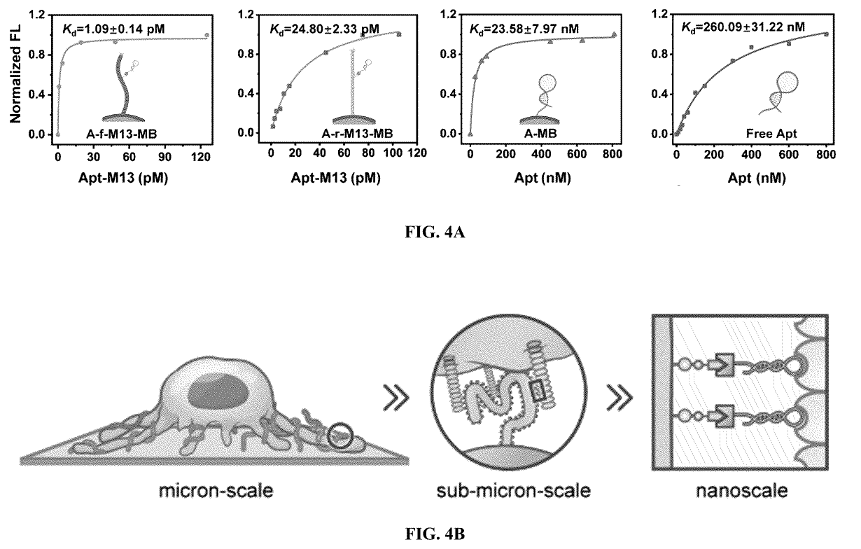

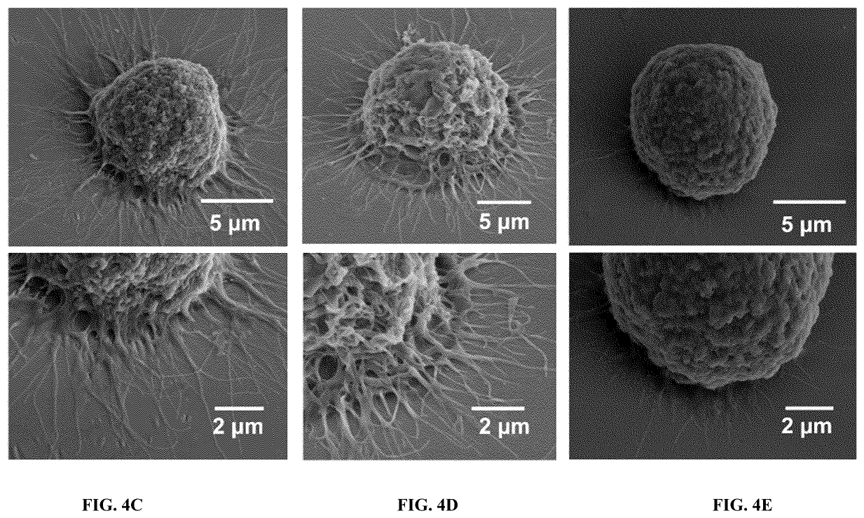

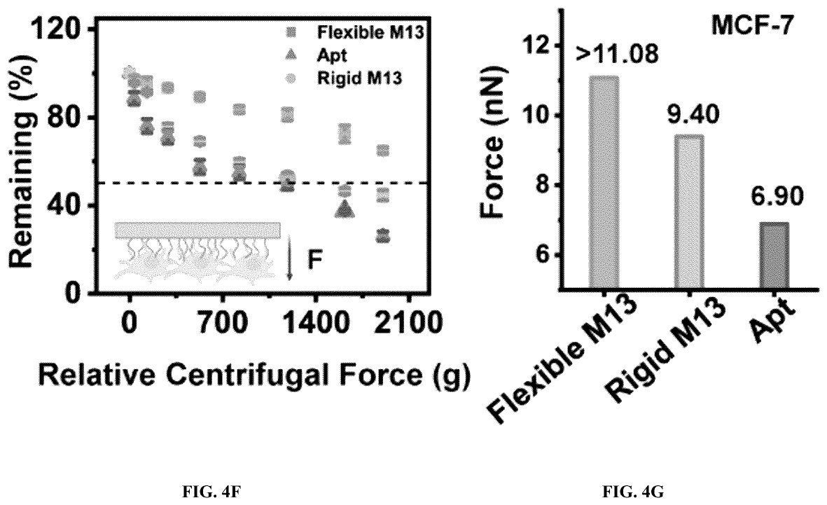

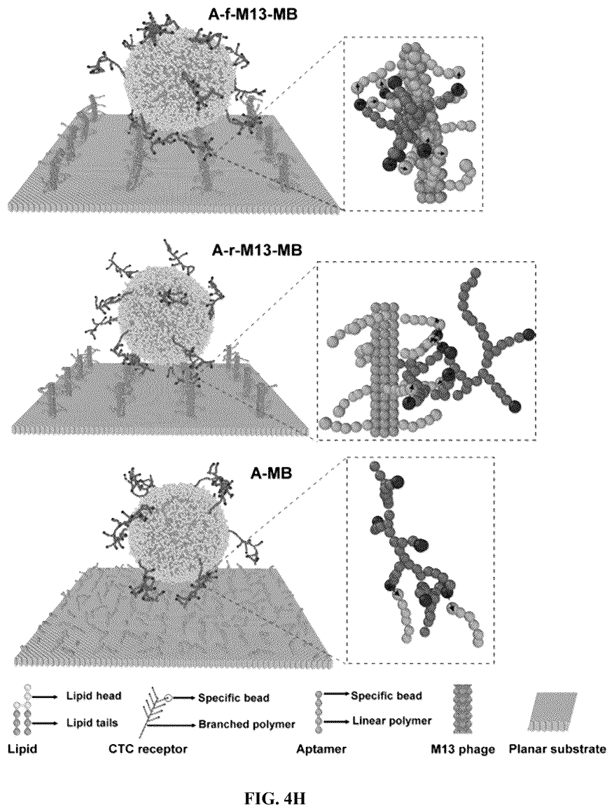

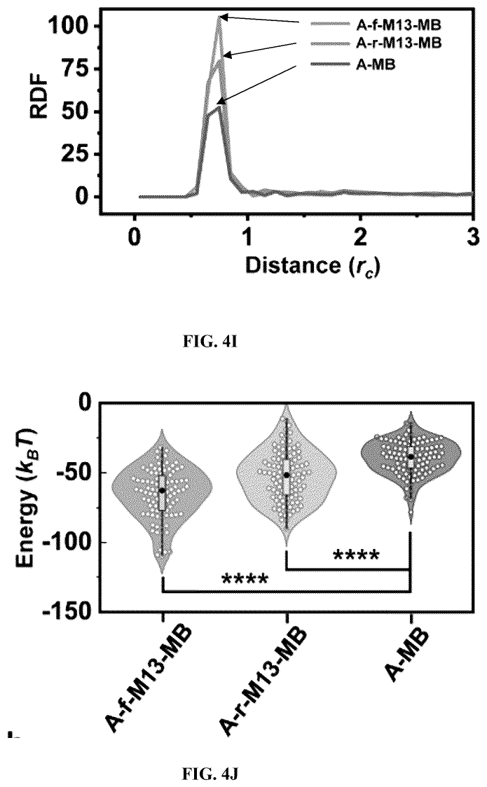

In this Example, MUC1 was chosen as the representative target receptor. MUC1 is a transmembrane glycoprotein that is often overexpressed in various epithelial cancers. Its extracellular domain extends around 200-500 nm beyond the cell surface, and is characterized by 20-120 VNTR repeats located in the N-terminal domain (Nath S. & Mukherjee, P. Trends in Mol. Med. 20, 332-342 (2014)). The elongated M13 nanofibers, spanning 880 nm and adorned with repeated VNTR-targeting aptamers, exhibit a favorable geometric fit with MUC1 (FIG. 4B). Although the aptamer distribution pattern might not achieve absolute alignment, the adaptable configuration of flexible M13 nanofibers promotes multivalent binding effect.

By securing the flexible Apt-M13 nanofibers onto Ni-IDA MBs to forge A-f-M13-MBs, the binding affinity to CTCs was assessed using MCF-7 as a model MUC1+CTC. Capitalizing on the molecular recognition between VNTR repeats and their corresponding aptamers, the sub-micron-scale fit between MUC1 and M13 nanofibers, and the micron-scale match between CTCs and M13-anchored topological interfaces (FIG. 4B), the adaptable CTC-capturing A-f-M13-MB (“A” for “aptamer” and “f” for “flexible”) surface exhibited an extraordinary 238,000-fold increase in CTC binding affinity (FIG. 4A, first panel on the left) compared to that of the unattached aptamer (FIG. 4A, first panel on the right). Furthermore, the CTC-binding affinity of the A-f-M13-MB surface exceeded that of the surface formed by directly immobilizing the aptamer onto MBs (i.e., A-MB) by approximately 21,600-fold. This underscores the pivotal role of the flexible Apt-M13 phage. Interestingly, substituting the flexible Apt-M13 phage with its rigid counterpart resulted in A-r-M13-MBs (“A” for “aptamer” and “r” for “rigid”) that displayed a substantial 22.8-fold decrease in CTC-binding affinity. Given that the aptamer loading level on MBs remained consistent (as shown in Table 1 below) and a comparable topological enhancement effect was observed for both A-f-M13-MBs and A-r-M13-MBs (FIGS. 4C-4E). The results show that flexibility of Apt-M13 is the primary driver behind increased CTC-binding affinity.

TABLE 1
The number of loaded aptamers on each MB
or M13 for flexible M13 and rigid M13.

| | Untreated | PFA-treated | EtOH-treated |
|---|---|---|---|
| | M13 | (rigid) M13 | (rigid) M13 |
| Amount of aptamers on | 1.04 | 1.26 | 0.7 |
| each MB (×10⁷) | | | |
| Number of aptamers on | 622 | 756 | 444 |
| each M13 | | | |

To investigate the role of M13 flexibility in the adhesive force between the CTC population and an affinity solid surface (e.g., MB or glass slide), a centrifugation-based cell adhesion assay was conducted as discussed in Reyes, C. D. and & Garci'a, A. J. J Biomed Mater Res. 67, 328-333 (2003) and Jiang, W. et al. Adv. Sci. 7, 2002259 (2020). In this setup, either flexible or rigid N3-M13 was affixed to Ni-IDA glass slides and equipped with aptamers. After a 30-minute WBC capture period on the M13 slides, a reverse centrifugal force was applied to the M13 slides with attached WBCs. As shown in FIGS. 4F-4G, flexible M13 exhibited a minimum of 18% higher adhesion force than its rigid counterpart. This outcome underscores once again that the inherent flexibility of M13 is instrumental in achieving robust binding to receptors on CTCs.

To gain deeper physical insights into the augmented multivalent interaction owing to flexibility, dissipative particle dynamics (DPD) simulations were performed to explore the intricate molecular-level interactions between CTCs and MBs. DPD simulations are discussed above in Example 1 and in Groot, R. D. and Warren, P. B. J. Chem. Phys. 107, 4423-4435 (1997). In the simulations, three distinct types of MBs, namely A-MB, A-f-M13-MB, and A-r-M13-MB, were constructed. As illustrated in FIG. 411, CTCs could be captured on all three MB surfaces; however, the underlying microscopic interaction modes differed significantly. For instance, in the case of A-MB, an individual CTC receptor could interact with only one or two aptamers located near the binding site. In contrast, in the case of A-r-M13-MB, due to the presence of multiple aptamers on each M13 phage, a single CTC receptor could simultaneously engage with numerous aptamers. The most intricate scenario unfolded with A-f-M13-MB, where the flexibility of the M13 phage allowed it to conform or twist to accommodate the receptor distribution on CTCs. Consequently, a CTC receptor could concurrently interact with a significantly larger number of aptamers. The micro-level distinction in interactions was further manifested in the radial distribution function (RDF) profiles. Specifically, the RDF peak values were approximately 105, 79, and 52 for A-f-M13-MB, A-r-M13-MB, and A-MB, respectively (FIG. 4I). The total energy of receptor-aptamer interaction in ascending order is from A-f-M13-MB (−65 k_{B}T), to A-r-M13-MB (−52 k_{B}T), and then to A-MB (˜−39 k_{B}T) (FIG. 4J). This order indicates that the interaction between CTCs and A-f-M13-MB was the most energetically favorable, likely due to the increased contacts between the M13-anchored aptamers and CTC receptors compared to the other two MB types.

It was worth noting that the freedom of M13 phage and/or the aptamers was restricted upon CTC adsorption on the MB surface (FIG. 4K). This reduction increased the entropy term (−TΔS) for the interaction, creating an entropic deterrent to adsorption. However, given the pronounced strength of the specific receptor-aptamer interaction, the interaction energy prevailed over these entropic factors during the adsorption process. This dynamic favored the interaction by causing a decline in the energy term that outweighed the concurrent rise in the entropy term.

### Example 5—Twisty M13 Facilitates Less White Blood Cell (WBC) Adsorption

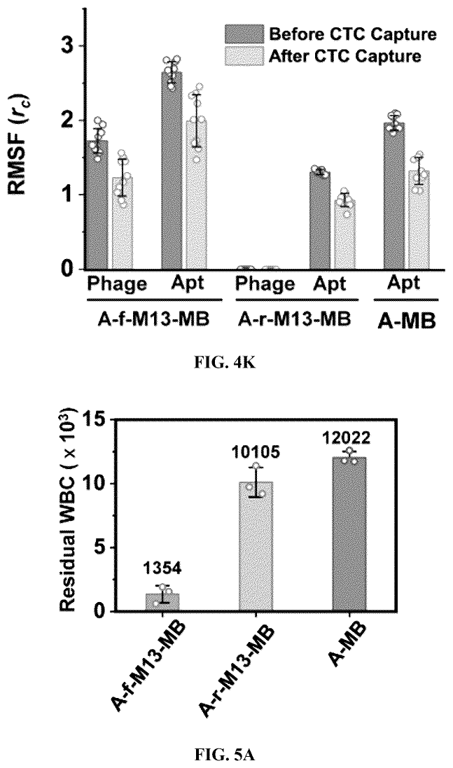

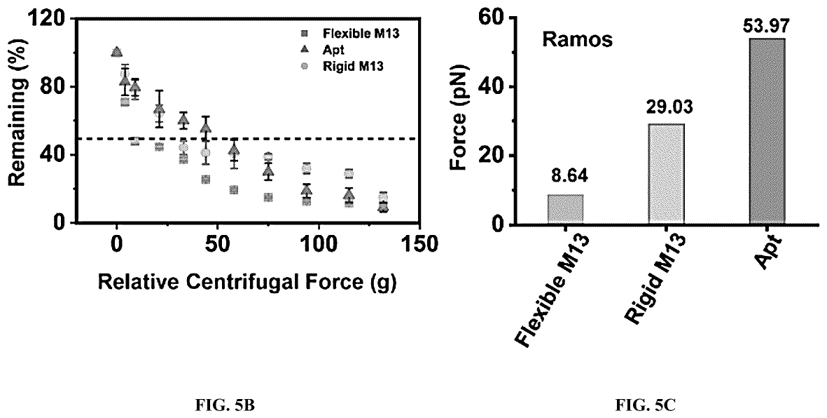

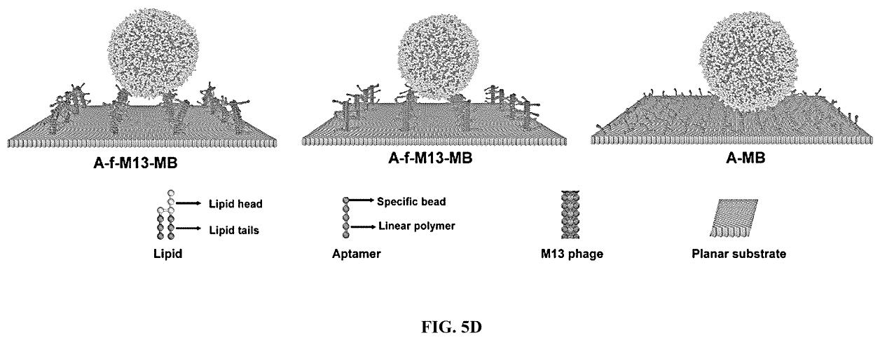

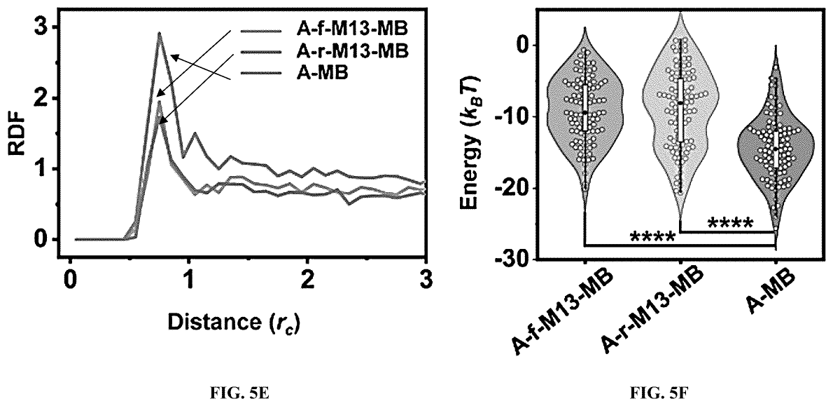

The exceptional scarcity of CTCs demands affinity surfaces with both high CTC binding affinity and robust anti-fouling capabilities. To assess the anti-fouling performance of the engineered surfaces, human Burkitt's lymphoma Ramos cells were used as a model for WBCs. Both flexible and rigid M13 nanofibers, each bearing the same level of aptamers, were anchored to Ni-IDA MBs. These MBs were subsequently exposed to 10⁶ Ramos cells. Notably, the outcomes revealed that only about ˜1354 WBCs adhered to the A-f-M13-MBs following a 30-minute incubation. In stark contrast, the number of WBCs binding to A-r-M13-MBs and A-MBs was 7.5-fold and 8.9-fold higher, respectively (FIG. 5A).

Further, results from the centrifugation-based cell adhesion assay disclosed a substantially reduced WBC binding force of 8.64 pN for A-f-M13-MBs. In this assay, either flexible N3-M13, rigid N3-M13, or aptamer (A) were anchored on Ni-IDA glass slides and loaded with aptamers. WBCs were first incubated with M13 slides for 30 min, and a reversed centrifugation force was then applied on the WBC-attached slide for 5 min. Remarkably, this centrifugal force accounted for merely one-third and one-sixth of the binding forces observed for A-r-M13-MBs and A-MBs, respectively (FIGS. 5B-5C).

For deeper insight into the distinct anti-fouling characteristics of the three surfaces at the molecular level, DPD simulations were performed. The modeled WBC, as shown in FIG. 5D, demonstrated a propensity for non-specific adsorption onto all three MB surfaces. Given the absence of specific receptor-ligand interactions, the contact between WBCs and MBs was significantly weaker, with the RDF peak registering at approximately 2-3 (FIG. 5E) compared to the more pronounced CTC-MB contacts (peaking within the 50-105 range, as seen in FIG. 4I). RDF indicates the relative density of aptamers around receptors. Higher RDF corresponds to a lower total energy of receptor-aptamer interaction. Notably, the RDF peak for A-MB was the highest, while the interaction energy was the lowest (FIG. 5F), thus rendering WBC adsorption energetically most favorable for A-MB. On the other hand, the RDF peak and interaction energy were nearly identical for A-f-M13-MB and A-r-M13-MB, implying similar energetic probabilities for WBC adsorption. However, the freedom of both M13 phage and ligands was restricted upon WBC adsorption to the MB surface (FIG. 5G). Importantly, for A-f-M13-MB, both M13 phage and aptamer freedoms were diminished, while for A-r-M13-MB, only aptamer freedom was curtailed. Consequently, the entropy term (−TΔS) was higher for A-f-M13-MB than for A-r-M13-MB. Of greater significance, given the relatively weak non-specific interaction (manifesting as a total interaction energy of several k_{B}T), the entropy term carried more weight than the energy term in dictating the interaction free energy. This dynamic resulted in less entropically favorable WBC adsorption onto the A-f-M13-MB surface than A-r-M13-MB.

### Example 6—Isolation of CTCs by M13-Anchored Microbeads (M13-MBs)

Having elucidated the underlying mechanism behind the flexibility-driven high-affinity binding and effective prevention of bio-fouling, the performance of aptamer-anchored flexible M13-MBs (A-f-M13-MB) for CTC isolation was evaluated under physiological conditions (FIG. 1A, Step (v)).

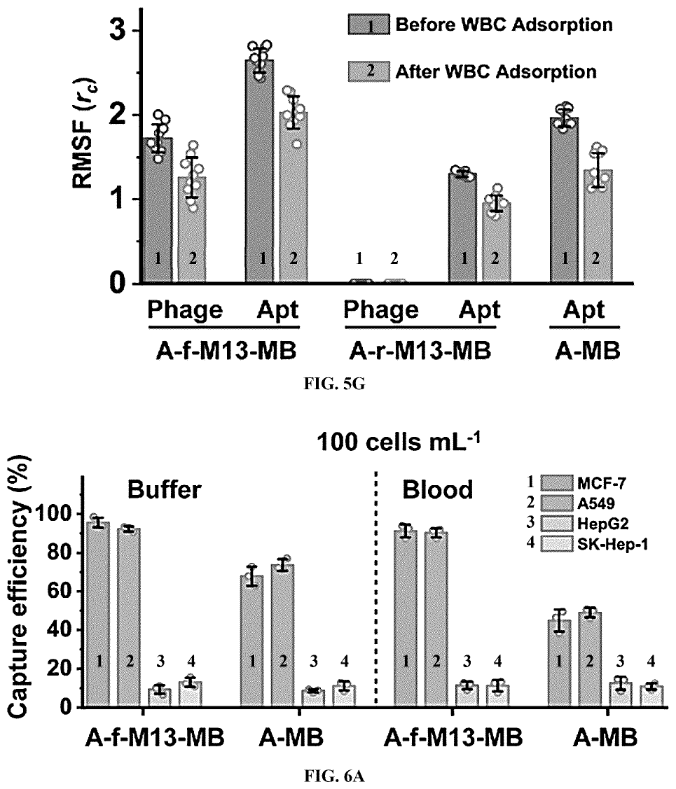

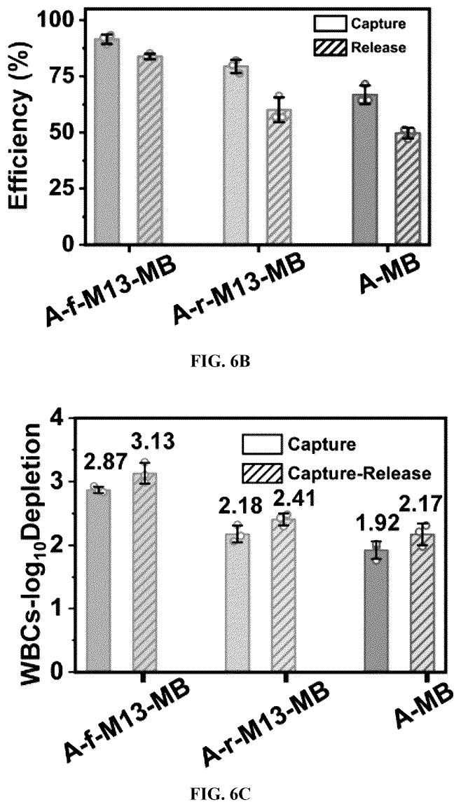

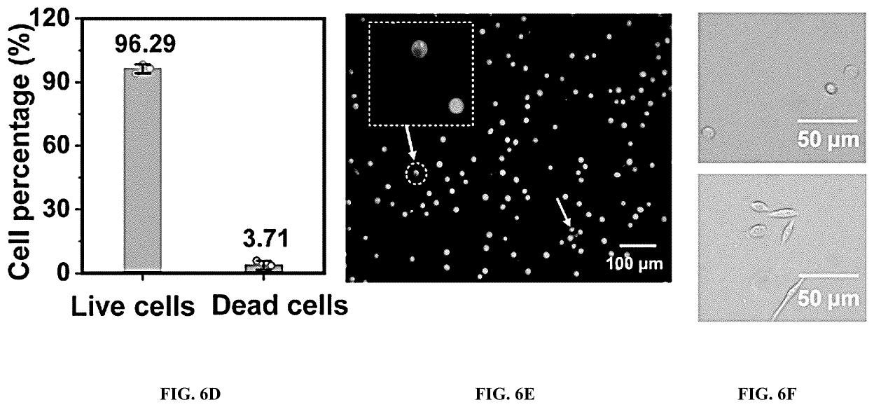

First, the aptamer was confirmed to bind MUC1-positive cells, including MCF-7 and A549 cancer cell lines (not shown). Then, selectivity of A-f-M13-MB for CTC capture was gauged by incubating A-f-M13-MB with MCF-7, A549, SK-Hep-1, and HepG2 cancer cells for 30 minutes (FIG. 6A, left panel). Notably, as illustrated in FIG. 6A, A-f-M13-MB successfully captured over 92.21% of MUC-1 positive cancer cells (MCF-7, A549), whereas the capture efficiencies for negative cells (HepG2, SK-Hep-1) were markedly lower, falling below 13.01%. This discrepancy underscores the impressive CTC-capture selectivity achieved by A-f-M13-MB. Furthermore, the efficiency of CTC capture by A-f-M13-MB remained scarcely affected in whole blood conditions, maintaining a capture rate exceeding 90.25% (FIG. 6A, right panel). Typically, aptamers are susceptible to degradation by nucleases present in whole blood, which significantly hampers their isolation performance in clinical settings. In this case, it is possible that the numerous aptamers on M13 nanofibers have the capacity to compensate for degraded aptamers nearby, and they might dynamically adjust their positions by freely twisting on the M13 scaffold. Consequently, this mechanism upholds high capture efficiency through multivalent binding, facilitated by a statistically rebinding mechanism.

Further, the CTC capture and release efficiency of A-f-M13-MB, A-r-M13-MB, and A-MB were compared using MCF-7 as the model cancer cell. Initially, MCF-7 cells were incubated with A-f-M13-MB for 30 minutes, magnetically isolated, rinsed, and subsequently released through DNase I digestion for microscopic enumeration (FIG. 1A, Step (vi)). As depicted in FIG. 6B, A-f-M13-MB achieved a capture efficiency of 91.46% for MCF-7 cells, which was approximately 12.08% higher than A-r-M13-MB and 24.67% higher than A-MB. Simultaneously, the flexible twisting of M13 nanofibers afforded sufficient interspace for DNase I access, resulting in a 23.74% improvement in cell release efficiency for A-f-M13-MB compared to A-r-M13-MB. This discrepancy in CTC release efficiency was even more pronounced when comparing A-f-M13-MB to A-MB (83.85% for A-f-M13-MB versus 49.73% for A-MB). In the case of A-MB, where aptamers were directly immobilized on the surface, DNase I struggled to reach the aptamer binding site once it was blocked post-CTC binding. Furthermore, CTC release performance remained nearly identical between different whole blood samples and buffer solutions, ensuring practical applicability in clinical scenarios.

Maintaining the purity and viability of released CTCs is paramount for downstream analysis. The flexible M13 nanofibers exhibit resistance to WBC attachment, leading to a relatively higher log 10-depletion index of WBCs for A-f-M13-MB than for A-r-M13-MB and A-MB (FIG. 6C). Since most WBCs are adsorbed non-specifically onto MBs without selective capture by aptamers, the DNase I-based strategy exclusively releases CTCs. This contributes to an increased WBC depletion index through the selective release of CTCs. Additionally, DNase I hydrolyzes the aptamers rather than degrading cells, ensuring high viability of the released CTCs, as confirmed by PI/AO staining (FIGS. 6D-6E, viability>96.29%). The isolated CTCs were successfully cultivated in 96-well plates and demonstrated steady adherence to the well surface over a 36-hour period (FIG. 6F). The migration ability and the phenotype drifting profile after the CTCs were released and re-cultured were also evaluated. The expression level of estrogen receptor protein (ER, feature biomarker of MCF-7 cells) showed no difference after CTC isolation and re-culture, indicating no phenotype drifting occurred after treatment with A-f-M13-MB. Namely, the isolated CTCs can faithfully represent the phenotypic feature of the original ones. These results indicate that after isolation and re-culture, the isolated CTCs have comparable or even better migration ability compared to the original CTCs. The high viability of the isolated CTCs can well meet the requirement for downstream applications including in vitro CTCs culturing or mouse transplantation xenograft construction.

### Example 7—CTC Isolation and Classification of Subtypes of Clinical Specimens Using M13-MBs

Breast cancer (BC) stands as the most prevalent malignant tumor and ranks second in terms of cancer-related mortality among women globally (Cao, Y. et al. J. Am. Chem. Soc. 143, 16078-16086 (2021)). This disease exhibits significant heterogeneity, categorized into distinct subtypes including luminal, human epidermal growth factor receptor 2 (HER2)-positive, and basal-like subtypes. Notably, patients with the luminal subtype generally experience a more favorable prognosis than the other subtypes. See, e.g., Tyanova, S. et al. Nat, Commun. 7, 10259 (2016); Yeo, S. K. and Guan, J. L. Trend in Cancer 3, 753-760 (2017); and Britt, K. L. et al. Nat Rev Cancer. 20, 417-436 (2020). Differential BC subtypes necessitate tailored treatments, indicating the urgency for the development of a rapid yet precise early-stage subtyping diagnostic approach.

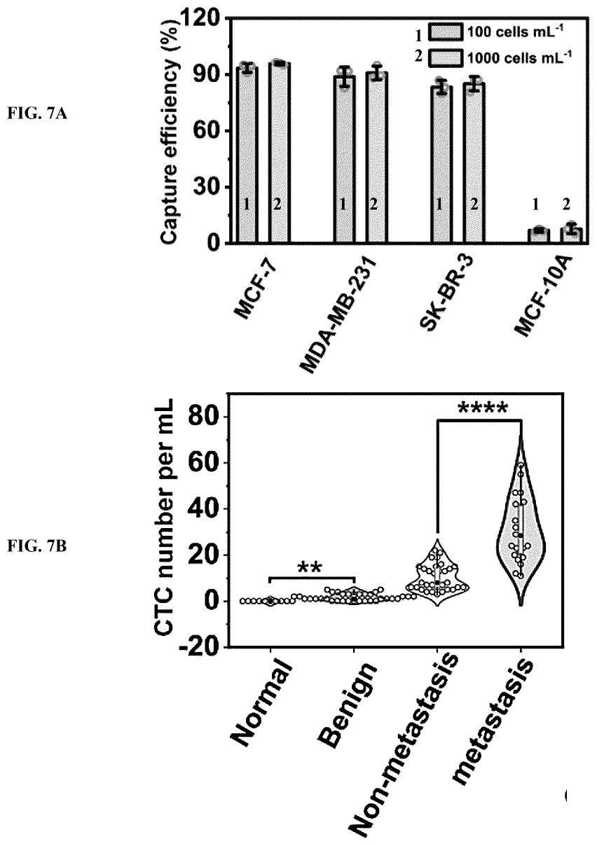

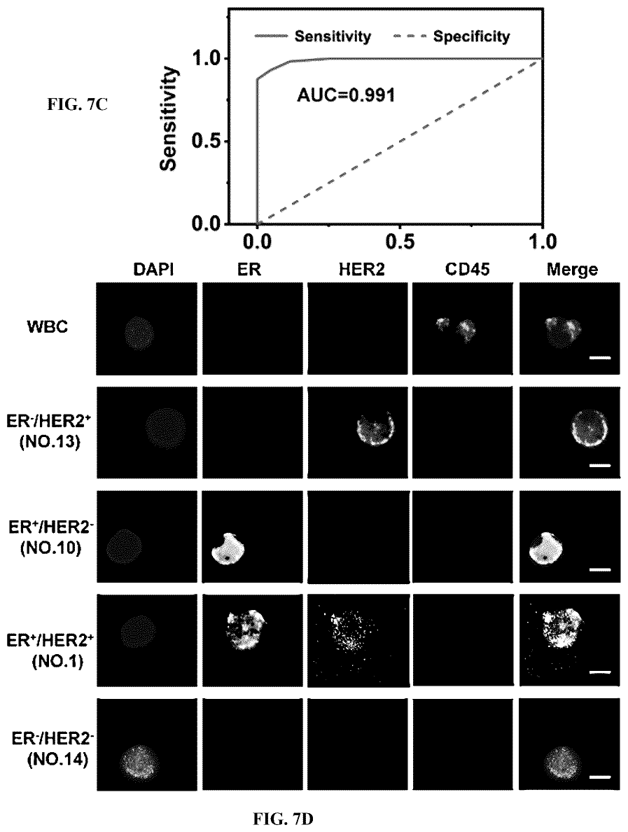

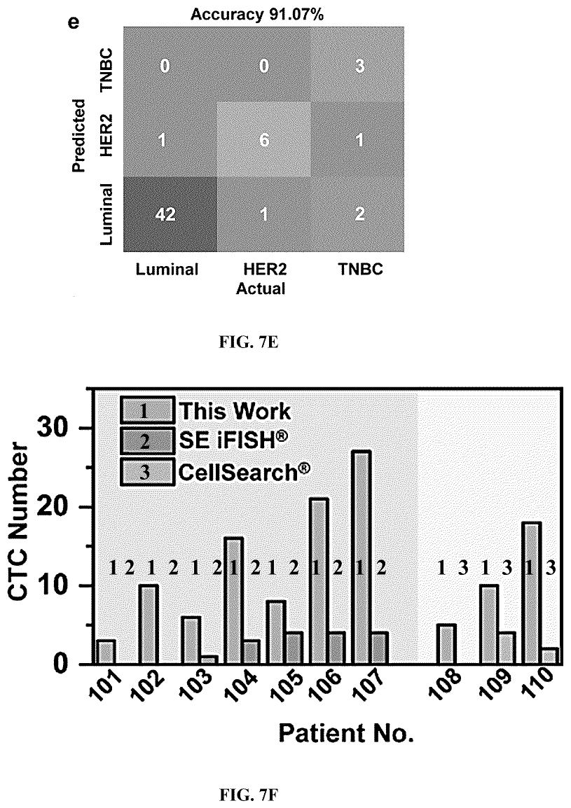

MUC1, excessively overexpressed in over 90% of breast tumors and 60% of captured circulating tumor cells (CTCs) from metastatic breast, lung, pancreatic, and colon cancer patients, holds promise as a biomarker for BC CTCs. See, e.g., Yin, X. et al. Anal. Chem. 92, 10308-10315 (2020); Kufe, D. W. Oncogene 32, 1073-1081 (2013); and Horm, T. M. and Schroeder, J. A. Cell Adhes. Migr. 7, 187-198 (2013). The feasibility of isolating BC CTCs using A-f-M13-MBs was assessed. Before translating to clinical samples, it was vital to evaluate the capturing efficacy of A-f-M13-MBs using model cells representing these BC subtypes. CTCs that were captured were released by DNase I and immuno-stained for the profiling of surface proteins including estrogen receptor protein (ER) and human epidermal growth factor receptor 2 (HER2) (FIG. 1B, Step (vi)-(vii). Immunofluorescence staining confirmed MUC1⁺/HER2⁻/estrogen receptor protein (ER)⁺ MCF-7 cells as luminal subtypes, MUC1⁺/HER2⁺/ER⁻ SK-BR-3 cells as HER2-positive subtypes, MUC1⁺/HER2⁻/ER⁻ MDA-MB-231 cells as basal-like subtypes, and MUC1⁻/HER2⁻/ER⁻ MCF-10A cells as normal breast mammary epithelial cells. Since these model BC cells were MUC1-positive, A-f-M13-MB consistently captured them with efficiencies ranging from 83.34% to 93.51% (FIG. 7A). However, non-specific capture of the normal cell line MCF-10A was a mere 6.99%, indicating the effective CTC selectivity of A-f-M13-MB in distinguishing BC cells from normal breast epithelial cells (FIG. 7A). Given that ER/HER2 expression significantly varies across subtypes, subtyping could be logically confirmed through immunofluorescence staining.

Having established nearly identical capture efficiency across different BC subtype CTCs, the diagnostic efficacy of A-f-M13-MB was assessed by analyzing blood samples from 56 BC patients, 34 benign patients, and 10 healthy volunteers without any prior treatment. Each assay involved evaluating 0.5 mL of whole blood, followed by CTC capture/release processes and a standard three-color immunocytochemistry staining protocol (Wang, S. et al. Anal. Chem. 94, 9450-9458 (2022)). Cells exhibiting CD45 negativity, DAPI positivity, and CK positivity (DAPI⁺/CK⁺/CD45⁻) were recognized as CTCs, while those staining positive for CD45 and DAPI but negative for CK (DAPI⁺/CK⁻/CD45⁺) were identified as white blood cells (WBCs). It was noted that the EpCAM-independent isolation strategy, which involves recognizing MUC1, facilitated the capture of EpCAM-negative CTCs from metastatic BC patients. This approach enhances clinical utility, especially for EpCAM-negative CTCs undergoing epithelial-mesenchymal transition (EMT) that often exhibit heightened metastatic potential. Patient No. 16, 72 and 88's severe lymphatic metastasis situation particularly underscores this point. As illustrated in FIG. 7B, CTCs were successfully identified and isolated from all BC patients, ranging from 3 to 59 CTCs per 1 mL of blood. In contrast, no more than 5 CTCs were found in blood samples from healthy donors or benign patients. CTC enumeration also facilitated differentiation between metastatic and non-metastatic BC patients. The receiver operating characteristic (ROC) curve exhibited promising diagnostic performance, with a sensitivity of 92.86%, specificity of 95.45%, and an area under the curve (AUC) of 0.991, with an optimal cutoff value of >4 CTC mL⁻¹ (FIG. 7C). Among the 56 patients sharing the same clinical diagnosis, the molecular profiling of isolated CTCs was analyzed by assessing ER and HER2 expression levels (FIG. 7D). As presented in FIG. 7E, 51 out of 56 patients were accurately classified into the correct subtype, achieving a diagnostic accuracy of 91.07%.

CTC isolation with A-f-M13-MB was compared to CTC capture using two different benchmark methods—the only FDA-approved CellSearch® method and another prevalent SE iFISH® technique. CellSearch® and SE iFISH® collected CTC from 7.5 mL and 6.0 mL blood, respectively, whereas the A-f-M13-MB approach (“This Work”) isolated CTC from 1.0 mL blood. (FIG. 7F). Due to the adaptive fitting multivalent binding of M13, the A-f-M13-MB approach demonstrated a significant increase in the number of isolated CTCs compared to CellSearch® and SE iFISH®. This enhancement significantly reduces the misdetection rate and false negatives associated with A-f-M13-MB.

### Example 8—Isolation of CTCs with Different Epithelial-Mesenchymal Transition (EMT) Subphenotypes by M13-MBs

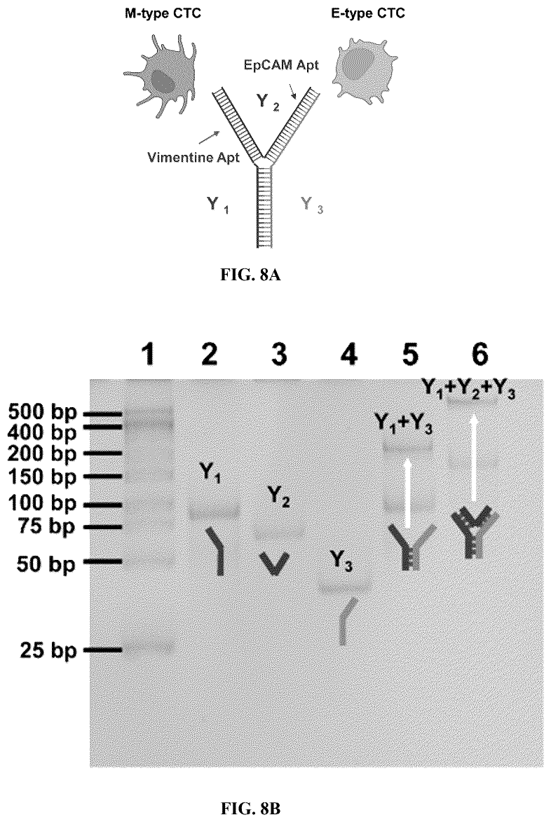

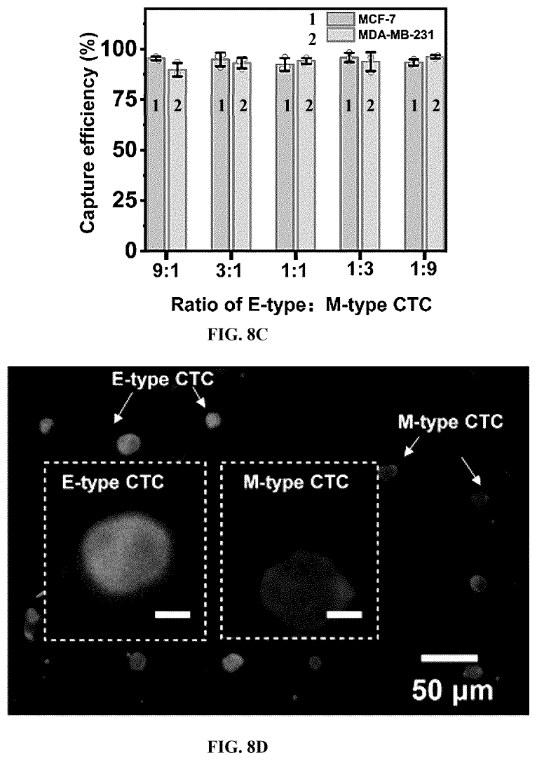

The existence of various CTCs with different EMT subphenotypes in cancer patient's blood, especially those suffering from metastasis, highlighted the importance to develop efficient CTC capture interfaces covering different EMT subphenotypes. Thus, a Y-shaped DNA scaffold was constructed to replace the MUC1 aptamer, to better isolate CTCs with different EMT characteristics. The Y-shaped DNA scaffold was assembled using an anti-vimentine aptamer (targeting the M-type CTC; Zheng, Y. et al. Anal. Chem. 92, 5178-5184 (2020)) and an anti-EpCAM aptamer (targeting the E-type CTC; Song, Y. et al. Anal. Chem. 85, 4141-4149 (2013)). See FIGS. 8A-8B for assembly detail; see the Informal Sequence Listing below for DNA sequences), and then attached to M13-MB via the same procedure described in Example 1 above under the subheading “Aptamer-loading on N3-M13 phage-immobilized magnetic microbeads (MBs)”.

For evaluating the CTC isolation performance of the resultant A-f-M13-MB (FIGS. 8A-8B), the two CTC model cells, E-type (MCF-7 cell, EpCAM⁺, N-cadherin⁻) and M-type (MDA-MB-231 cell, EpCAM⁻, N-cadherin⁺), were mixed with different ratios and added to CTC-free whole blood. A-f-M13-MB was added to the mixture. Isolation of CTCs were performed and the isolated CTCs were subjected to immunofluorescence staining for phenotyping. As shown in FIG. 8C, both cell types were captured at high efficiency regardless of the ratio of E-type/M-type, and the subphenotypes were clearly identified by immunofluorescence images (FIG. 8D). This underscored the ability of A-f-M13-MB to isolate and identify CTCs with different EMT characteristics.

### CONCLUSION

This study reveals the significant role played by the mechanical attributes of virus-modified solid surfaces in the isolation of rare cells, such as CTCs, with respect to both target cell binding affinity and the mitigation of non-specific interactions with non-target cells like WBCs. The inherent flexibility and deformability of virus nanofibers confer a self-adjusting capability to the solid surface, thereby amplifying multivalent interactions between the aptamers residing on the nanofibers and the receptors on target cells through energy-driven processes. Additionally, these flexible virus nanofibers on the solid surface yield a marked reduction in the non-specific adsorption of non-target cells when compared to their rigid counterparts, attributed to an entropy-driven mechanism resulting from the greater loss of freedom within flexible nanofibers relative to rigid ones. The approach discussed in these Examples, reliant on flexible virus nanofibers, outperforms conventional methods based on anti-fouling polymers, as it avoids the potential drawbacks linked to such polymers and simultaneously provides the necessary space on the solid surface for the specific ligand recognition of target cells. This phage-based strategy not only introduces an affinity-based solid bioassay but also draws upon a deeper mechanistic understanding of target-ligand interactions, thereby enhancing the efficiency of CTC isolation.

### REFERENCES

The following references relate to the Examples.
1. Wu, L. et al. Fluidic multivalent membrane nanointerface enables synergetic enrichment of circulating tumor cells with high efficiency and viability. J. Am. Chem. Soc. 142, 4800-4806 (2020).
2. Jeong, W. J. et al. Hierarchically multivalent peptide-nanoparticle architectures: a systematic approach to engineer surface adhesion. Adv. Sci. 9, 2103098 (2022)
3. Guo, L. et al. Directing multivalent aptamer-receptor binding on the cell surface with programmable atom-like nanoparticles. Angew. Chem. Int. Ed. 61, e202117168 (2022).
4. Sabaté del Rio, J., Henry, O. Y F., Jolly P. & Ingber, D. E. An antifouling coating that enables affinity-based electrochemical biosensing in complex biological fluids. Nat. Nanotechnol. 14, 1143-1149 (2019).
5. Banerjee, I., Pangule, R. C. & Kane, R. S. Antifouling coatings: recent developments in the design of surfaces that prevent fouling by proteins, bacteria, and marine Organisms. Adv. Mater. 23, 690-718 (2011).
6. Jiang, C., Wang, G., Hein, R., Liu, N., Luo, X. & Davis, J. J. Antifouling strategies for selective in vitro and in vivo sensing. Chem. Rev. 120, 3852-3889 (2020).
7. Yuan, Z. et al. Zwitterionic peptide cloak mimics protein surfaces for protein protection. Angew. Chem. Int. Ed. 59, 22378-22381 (2020).
8. Li, C., Lu, D., Deng, J., Zhang, X. & Yang, P. Amyloid-like rapid surface modification for antifouling and in-depth remineralization of dentine tubules to treat dental hypersensitivity. Adv. Mater. 31, 1903973 (2019).
9. Maan, A. M. C., Hofman, A. H., M. de. Vos, W. & Kamperman, M. Recent developments and practical feasibility of polymer-based antifouling coatings. Adv. Funct. Mater. 30, 2000936 (2020).
10. Nowinski, A. K., Sun, F., White, A. D., Keefe, A. J. & Jiang, S. Sequence, structure, and function of peptide self-assembled monolayers. J. Am. Chem. Soc. 134, 6000-6005 (2012).
11. Song, Z., Chen, M.; Ding, C. & Luo, X. Designed three-in-one peptides with anchoring, antifouling, and recognizing capabilities for highly sensitive and low-fouling electrochemical sensing in complex biological media. Anal. Chem. 92, 5795-5802 (2020).
12. Tian, Y et al. Engineering poly(ethylene glycol) nanoparticles for accelerated blood clearance inhibition and targeted drug delivery. J. Am. Chem. Soc. 144, 18419-18428 (2022).
13. Liu, X. et al. Highly effective stroke therapy enabled by genetically engineered viral nanofibers. Adv. Mater. 34, 2201210 (2022).
14. Zeng, Y, Yue, H., Cao, B., Li, Y, Yang, M. & Mao, C. Target-triggered formation of artificial enzymes on filamentous phage for ultrasensitive direct detection of circulating miRNA biomarkers in clinical samples. Angew. Chem. Int. Ed. 61, e202210121 (2022).
15. Chang, C. et al. Engineered M13 phage as a novel therapeutic bionanomaterial for clinical applications: From tissue regeneration to cancer therapy. Materials Today Bio 20, 100612 (2023).
16. Cao, B., Li, Y, Yang, T., Bao, Q., Yang, M. & Mao, C. Bacteriophage-based biomaterials for tissue regeneration. Advanced Drug Delivery Reviews. 145, 73-95 (2019).
17. Zhou, N. et al. Hierarchical ordered assembly of genetically modifiable viruses into nanoridge-in-microridge structures. Adv. Mater. 31, 1905577 (2019).
18. Tsedev, U., Lin, C. W., Hess, G. T, Sarkaria, J. N., Lam, F. C. & Belcher, A. M. Phage particles of controlled length and genome for In vivo targeted glioblastoma imaging and therapeutic delivery. ACS Nano 16, 11676-11691 (2022).
19. Van Nieuwenhuyse, B. et al. Bacteriophage-antibiotic combination therapy against extensively drug-resistant Pseudomonas aeruginosa infection to allow liver transplantation in a toddler. Nature Communications. 13, 5725 (2022).
20. Peng, H., Borg, R. E., Dow, L. P., Pruitt, B. L. & Chen, I. A. Controlled phage therapy by photothermal ablation of specific bacterial species using gold nanorods targeted by chimeric phages. Proc. Natl. Acad. Sci. U.S.A, 117, 1951-1961 (2022).
21. Mao, C., Liu, A. & Cao, B. Virus-based chemical and biological sensing. Angew. Chem. Int. Ed. 48, 6790-6810 (2009).
22. Oh, J. W. et al. Biomimetic virus-based colourimetric sensors. Nat. Commun. 5, 3043 (2014).
23. Lee, B. Y et al. Virus-based piezoelectric energy generation. Nature Nanotech. 7, 351-356 (2012).
24. Cao, Y et al. Efficient pathogen capture and sensing promoted by dynamic deformable nanointerfaces. Small 18, 2203962 (2022).
25. Fidler, I. J. The pathogenesis of cancer metastasis: the ‘seed and soil’ hypothesis revisited. Nat Rev Cancer. 3, 453-458 (2013).
26. Brechot, P. P. & Benali, N. L. Circulating tumor cells (CTC) detection: clinical impact and future directions. Cancer Letters. 253, 180-204 (2007).
27. Wu, L. et al. Beyond capture: circulating tumor cell release and single-cell analysis. Small Methods 3, 1800544 (2019).
28. Song, Y et al. Enrichment and single-cell analysis of circulating tumor cells. Chem. Sci., 8, 1736-1751 (2017).
29. Wu, L. et al. Aptamer-based detection of circulating targets for precision medicine. Chem. Rev. 121, 12035-12105 (2021).
30. Luo, D. et al. Biocompatibility of magnetic resonance imaging nanoprobes improved by transformable Gadolinium Oxide nanocoils. J. Am. Chem. Soc. 140, 14211-14216 (2018).
31. Ferreira, C. S. M., Matthews, C. S. & Missailidis, S. DNA aptamers that bind to MUC1 tumor marker: design and characterization of MUC1-binding single-stranded DNA aptamers. Tumor Biol 27, 289-301 (2006).
32. Chen, L., Wu, Y, Lin Y & Wang, Q. Virus-templated FRET platform for the rational design of ratiometric fluorescent nanosensors. Chem. Commun., 51, 10190-10193 (2015).
33. Fasting, C. et al. Multivalency as a chemical organization and action principle. Angew. Chem. Int. Ed. 51, 10472-10498 (2012).
34. Bhatia, S., Camacho, L. C. & Haag, R. Pathogen inhibition by multivalent ligand architectures. J. Am. Chem. Soc. 138, 8654-8666 (2016).
35. Bachem, G. et al. Rational design of a DNA-scaffolded high-affinity binder for langerin. Angew. Chem. Int. Ed. 59, 21016-21022 (2020).
36. Nath S. & Mukherjee, P. MUC1: a multifaceted oncoprotein with a key role in cancer progression. Trends in Mol. Med. 20, 332-342 (2014).
37. Reyes, C. D. & Garci'a, A. J. A centrifugation cell adhesion assay for high-throughput screening of biomaterial surfaces. J Biomed Mater Res. 67, 328-333 (2003).
38. Jiang, W. et al. Natural fish trap-like nanocage for label-free capture of circulating tumor cells. Adv. Sci. 7, 2002259 (2020).
39. Groot, R. D. & Warren, P. B. Dissipative particle dynamics: Bridging the gap between atomistic and mesoscopic simulation. J. Chem. Phys. 107, 4423-4435 (1997).
40. Cao, Y et al. In situ programmable DNA circuit-promoted electrochemical characterization of stemlike phenotype in breast cancer. J. Am. Chem. Soc. 143, 16078-16086 (2021).
41. Tyanova, S., Albrechtsen, R., Kronqvist, P., Cox, J., Mann, M. & Geiger, T. Proteomic maps of breast cancer subtypes. Nat, Commun. 7, 10259 (2016).
42. Yeo, S. K. & Guan, J. L. Breast cancer: multiple subtypes within a tumor?Trend in cancer. 3, 753-760 (2017).
43. Britt, K. L., Cuzick, J. & Phillips, K. A. Key steps for effective breast cancer prevention. Nat Rev Cancer. 20, 417-436 (2020).
44. Yin, X., Chen, B., He, M. & Hu, B. A multifunctional platform for the capture, release, and enumeration of circulating tumor cells based on aptamer binding, nicking endonuclease-assisted amplification, and inductively coupled plasma mass spectrometry detection. Anal. Chem. 92, 10308-10315 (2020).
45. Kufe, D. W. MUC1-C oncoprotein as a target in breast cancer: activation of signaling pathways and therapeutic approaches. Oncogene 32, 1073-1081 (2013).
46. Horm, T. M. & Schroeder, J. A. MUC1 and metastatic cancer. J. A. Cell Adhes. Migr. 7, 187-198 (2013).
47. Wang, S. et al. Reversible and highly ordered biointerfaces for efficient capture and nondestructive release of circulating tumor cells. Anal. Chem. 94, 9450-9458 (2022).
48. Zheng, Y et al. Selection of aptamers against vimentin for isolation and release of circulating tumor cells undergoing epithelial mesenchymal transition. Anal. Chem. 92, 5178-5184 (2020).
49. Song, Y et al. Selection of DNA aptamers against epithelial cell adhesion molecule for cancer cell imaging and circulating tumor cell capture. Anal. Chem. 85, 4141-4149 (2013).
50. Alarcó n-Correa, M. et al. Self-assembled phage-based colloids for high localized enzymatic activity. ACS Nano 13, 5810-5815 (2019).
51. Shen, W., Zhong, H., Neff, D. & L. Norton, M. NTA directed protein nanopatterning on DNA origami nanoconstructs. J. Am. Chem. Soc. 131, 6660-6661 (2009).
52. Conti, M., Falini, G & Samori, B. How strong is the coordination bond between a histidine tag and Ni-nitrilotriacetate?An experiment of mechanochemistry on single molecules. Angew. Chem. Int. Ed. 112, 221-224 (2000).
53. Yuan, R., Ge, F., Liang, Y., Zhou, Y., Yang, L. & Wang, Q. A virus-like element-tagged nanoparticle ICPMS signal multiplier: membrane biomarker mediated cell counting. Anal. Chem. 91, 4948-4952 (2019).
54. Jeon, C. S., Hwang, I. & Chung, T. D. Virus-tethered magnetic gold microspheres with biomimetic architectures for enhanced immunoassays. Adv. Funct. Mater. 23, 1484-1489 (2013).
55. Wu, N. et al. Three-dimensional DNA nanomachine biosensor by integrating DNA walker and rolling machine cascade amplification for ultrasensitive detection of cancer-related gene. Anal. Chem. 92, 11111-11118 (2020).
56. Liu, J., Yang, T., Chen, S., Chen, X. & Wang, J. Nickel chelating functionalization of graphene composite for metal affinity membrane isolation of lysozyme. J. Mater. Chem. B, 1, 810-818 (2013).
57. Alexeev, A., Uspal, W. E. & Balazs, A. C. Harnessing Janus nanoparticles to create controllable pores in membranes. ACS Nano 2, 1117-1122 (2008).
58. Ding, H. M., Tian, W. D. & Ma, Y. Q. Designing nanoparticle translocation through membranes by computer simulations. ACS Nano 6, 1230-1238 (2012).
59. Yang, K. & Ma, Y. Q. Computer simulation of the translocation of nanoparticles with different shapes across a lipid bilayer. Nat. Nanotechnol. 5, 579-583 (2010).
60. Plimpton, S. Fast parallel algorithms for short-range molecular dynamics. J. Comput. Phys. 117, 1-19 (1995).
61. Jo, S. M., Lee, J. J., Heu, W. & Kim, H. S. Nanotentacle-structured magnetic particles for efficient capture of circulating tumor cells. Small 11, 1975-1982 (2015).

All publications, issued patents, and patent applications cited in this specification are herein incorporated by reference as if each individual publication or patent application were specifically and individually indicated to be incorporated by reference.

It is to be understood that this disclosure is not limited to the particular methodology, protocols, cell lines, animal species or genera, and reagents described, as such may vary. It is also to be understood that the terminology used herein is for the purpose of describing particular embodiments only and is not intended to limit the scope of the present disclosure, which will be limited only by the appended claims.

INFORMAL SEQUENCE LISTING

| SEQ ID | | |
|---|---|---|
| NO | Description | Sequence |
| 1 | DBCO-Apt | DBCO-TTTTTGCAGTTGATCCTTTGGATACCCTGG |
| | aptamer | |
| | sequence; S2.2 | |
| | sequence | |
| 2 | FAM-Apt- | DBCO-TTTTTGCAGTTGATCCTTTGGATACCCTGG-FAM |
| | DBCO aptamer | |
| | sequence | |
| 3 | NH2-Apt | NH₂-TTTTTGCAGTTGATCCTTTGGATACCCTGG |
| | aptamer | |
| | sequence | |
| 4 | FAM-Apt-NH₂ | NH₂-TTTTTGCAGTTGATCCTTTGGATACCCTGG-FAM |
| | aptamer | |
| | sequence | |
| 5 | 6H-Tag1 | GTACCTTTCTATTCTCACTCTCATCATCATCATCATCATTCCT |
| | oligonucleotide | CCAAACTGCAGTC |
| | sequence | |
| 6 | 6H-Tag2 | GGCCGACTGCAGTTTGGAGGAATGATGATGATGATGATGA |
| | oligonucleotide | GAGTGAGAATAGAAAG |
| | sequence | |
| 7 | Y₁ | CACGCATAGCCTTTGCTCCTCGTCTGGAACGTCGCAGCTT |
| | oligonucleotide | TAGTTCTGGGCCTATGCGTGTTTTTTGTAGTCGGTACCTA |
| | sequence for Y- | AGACTTCTGAGCATGCACTGAC |
| | shaped DNA | |
| | scaffold | |
| 8 | Y₂ | CACTACAGAGGTTGCGTCTGTCCCACGTTGTCATGGGGG |
| | oligonucleotide | GTTGGCCTGTTTTTTGTCAGTGCATGCTCAGAAGAACTCA |
| | sequence for Y- | CGTGACG |
| | shaped DNA | |
| | scaffold | |
| 9 | Y₃ | DBCO- |
| | oligonucleotide | ATTGCGTATGTCACGTCACGTGAGTTCGTCTTAGGTACCG |
| | sequence for Y- | ACTAC |
| | shaped DNA | |
| | scaffold | |

1. A modified phage comprising on its surface an aptamer that specifically binds to a cancer-related antigen.

2. The modified phage of claim 1, wherein the aptamer comprises a sequence as set forth in SEQ ID NO:1.

3. The modified phage of claim 1, wherein the aptamer is covalently linked to a first capsid protein of the modified phage.

4. The modified phage of claim 1, wherein the modified phage is immobilized on a solid substrate via a second capsid protein of the modified phage.

5. The modified phage of claim 1, wherein the modified phage is a modified M13 phage.

6. The modified phage of claim 3, wherein the first capsid protein is pIII, pVI, pVII, pVIII, or pVIX.

7. The modified phage of claim 3, wherein the first capsid protein is pVIII.

8. The modified phage of claim 3, wherein the covalent link between the aptamer and the first capsid protein comprises an azide linker.

9. The modified phage of claim 4, wherein the second capsid protein is pIII, pVI, pVII, pVIII, or pVIX.

10. The modified phage of claim 4, wherein the second capsid protein is pIII.

11. The modified phage of claim 4, wherein the solid substrate is a magnetic bead.

12. A composition comprising the modified phage of claim 1 and a pharmaceutically acceptable excipient.

13. A method for isolating cancer cells comprising:
(a) contacting cancer cells with an effective amount of the modified phage of claim 1; and
(b) providing conditions that allow specific binding of the modified phage to the cancer cells;
thereby isolating the cancer cells by removing the modified phage.

14. The method of claim 13, further comprising releasing cancer cells that were bound to the modified phage.

15. The method of claim 13, further comprising identifying cancer cell subtype.

16. The method of claim 13, further comprising administering treatment to the subject based on the identified cancer cell subtype.

17. The method of claim 13, wherein the cancer cells are breast cancer cells, liver cancer cells, lung cancer cells, prostate cancer cells, colon and rectal cancer cells, stomach cancer cells, pancreatic cancer cells, esophageal cancer cells, kidney cancer cells, bladder cancer cells, brain cancer cells, or ovarian cancer cells.

18. A kit for isolating cancer cells comprising:
(a) the modified phage of claim 1; and
(b) instructions for using the kit.

19. The kit of claim 18, further comprising a magnet.
